(19) United States
(12) Patent Application Publication
(10) Pub. No.: US 2026/0000282 A1
(43) Pub. Date: Jan. 1, 2026

(54) SYSTEMS AND METHODS FOR PROVIDING MEDICAL FLUORESCENCE IMAGING USING A GLOBAL SHUTTER IMAGER AND A LIQUID CRYSTAL LIGHT SHUTTER

(71) Applicant: Stryker Corporation, Portage, MI (US)

(72) Inventors: Benjamin Hyman FEINGOLD, San Francisco, CA (US); Chien Mien PANG, San Jose, CA (US); Levey Trac TRAN, Denver, CO (US); Paul Roald WESTWICK, Vancouver (CA); Marc ANDRÉ, Spiegel b. Bern (CH)

(73) Assignee: Stryker Corporation, Portage, MI (US)

(21) Appl. No.: 19/249,873

(22) Filed: Jun. 25, 2025

Related U.S. Application Data
(60) Provisional application No. 63/664,664, filed on Jun. 26, 2024.

(51) Int. Cl. A61B 1/04; A61B 1/00; A61B 1/045; A61B 1/06; H04N 23/56

(52) U.S. Cl. CPC A61B 1/043; A61B 1/00009; A61B 1/045; A61B 1/0653; A61B 1/0655; H04N 23/56

(57) ABSTRACT

The present disclosure relates to techniques for imaging tissue of a subject. An exemplary method comprises transitioning the liquid crystal light shutter to the closed state to prevent the global shutter imager from receiving visible light; illuminating the tissue of the subject with the fluorescence excitation illumination source to accumulate charge at a plurality of pixels of the global shutter imager, while the liquid crystal light shutter is in the closed state; reading a set of accumulated charge at the plurality of pixels of the global shutter imager to produce a set of imaging data; and generating a fluorescence image frame based on the set of imaging data.

### CROSS-REFERENCE TO RELATED APPLICATION

This application claims the benefit of U.S. Provisional Application 63/664,664 filed on Jun. 26, 2024, the entire contents of which are incorporated herein by reference for all purposes.

### FIELD

The present disclosure relates generally to medical imaging, and more specifically to techniques for providing medical fluorescence imaging (e.g., open-field surgery fluorescence visualization) with a global shutter imager and a light shutter.

### BACKGROUND

Medical systems, instruments, or tools are utilized pre-surgery, during surgery, or post-operatively for various purposes. In particular, medical imaging systems can be used to enable a surgeon to view a surgical site in open-field procedures and endoscopic procedures. For example, endoscopy in the medical field allows internal features of the body of a patient to be viewed without the use of traditional, fully invasive surgery. Endoscopic imaging systems incorporate endoscopes to enable a surgeon to view a surgical site, and endoscopic tools enable non-invasive surgery at the site. Endoscopes may be usable along with a camera system for processing the images received by the endoscope. An endoscopic camera system typically includes a camera head connected to a camera control unit (CCU) that processes input image data received from the image sensor of the camera and outputs the image data for display. The CCU may control an illuminator or illumination source that generates illumination light provided to the imaged scene.

Medical imaging systems (e.g., endoscopic imaging systems, open-field imaging systems) may include both a visible-light illumination source (also referred to as white-light illumination source) and a fluorescence excitation illumination source. The visible-light illumination source and the fluorescence excitation illumination source can be used to illuminate a tissue of a subject to obtain different types of image data (e.g., fluorescence image frames, white-light image frames, blended image frames). In particular, the fluorescence excitation illumination source, such as an infrared light, can provide illumination to the imaged tissue to excite the fluorophores (or fluorochromes) to produce fluorescence emission.

Existing medical imaging systems using global shutter imagers have several deficiencies. In order to obtain clean fluorescence imaging data, some existing systems turn off the visible-light illumination source during the acquisition of the fluorescence imaging data. However, some ambient visible light still exists in the environment and thus can contaminate the fluorescence imaging data. To overcome this drawback, many existing imaging systems need to acquire ambient visible-light imaging data, which is subtracted from the fluorescence imaging data to obtain a final fluorescence image frame. The additional acquisition of ambient visible-light imaging data in order to acquire each fluorescence image frame increases the number of frame periods required to acquire each fluorescence image frame and thus reduces the output image update rate of the imaging systems.

Further, some existing imaging systems rely on a mechanical shutter to block all visible light signals during the acquisition of fluorescence imaging data. However, the mechanical shutter has a relatively slow response time and is not suitable for applications requiring rapid switching or modulation of light. As an example, one type of standard mechanical shutter of a medical imaging system may take approximately 500 milliseconds to switch between states. Thus, these imaging systems cannot be configured to obtain both fluorescence imaging data and corresponding visible-light imaging data, which significantly reduces their usability (e.g., for medical imaging purposes).

Thus, techniques for acquiring both fluorescence imaging data and corresponding visible-light imaging data using global shutter imagers are desirable.

### SUMMARY

Examples of the present disclosure include various examples of an illumination scheme that acquire both fluorescence imaging data and corresponding visible-light imaging data using global shutter imagers (e.g., for surgical imaging applications). The techniques described herein can provide an improved medical imaging system that can alternate between acquisition of clean fluorescence imaging data (e.g., uncontaminated from ambient visible light) and acquisition of visible-light imaging data. In particular, the clean fluorescence imaging data is acquired via the use of a light shutter to block visible light from reaching the sensor of the global shutter imager. Examples of the present disclosure can either provide continuous visible-light illumination or pulsed visible-light illumination, pulsing rapidly enough to avoid strobe effects and jerky motion.

The techniques described herein can utilize a light shutter, such as a liquid crystal (LC) shutter, for selective blocking of visible light from reaching the imager. The light shutter has an open state and a closed state and can transition between the two states based on a control signal for operating the light shutter, as described herein. In some examples, the light shutter is a liquid crystal (LC) shutter. The light shutter can behave differently for a light reflected from illumination of a tissue of the subject by the visible-light illumination source and a light emitted due to illumination of a tissue of the subject by the fluorescence excitation illumination source. Specifically, the light shutter can, in the open state, allow the passage of the light reflected from illumination of the tissue of the subject with the visible-light illumination such that the reflected light can reach the imager. In the closed state, the light shutter can prevent the light reflected from illumination of the tissue of the subject from reaching the imager or significantly attenuate the reflected light reaching the imager.

Further, regardless of which state the light shutter is in (e.g., the open state, the closed state, the transitioning state from the open state to the closed state, the transitioning state from the closed state to the open state), the light shutter can always allow the passage of a light emitted due to illumination of the tissue of the subject with the fluorescence excitation illumination source. In other words, the light shutter can transmit the fluorescence light emitted due to illumination of the tissue of the subject with the fluorescence excitation illumination even when the light shutter is in the closed state, so the opening and/or closing of the light shutter only affects the passage of the light reflected from illumination of the tissue of the subject with the visible-light illumination source. When the light shutter is in the closed state, no light reflected from illumination of the tissue of the subject with the visible-light illumination source is transmitted; instead, only the light emitted due to illumination of the tissue of the subject with the fluorescence excitation illumination source is transmitted.

In some examples of an illumination scheme, an exemplary system comprises a global shutter imager, a liquid crystal light shutter configurable to be in an open state and a closed state, and a fluorescence excitation illumination source. The system transitions the liquid crystal light shutter to the closed state to prevent the global shutter imager from receiving visible light and illuminates the tissue of the subject with the fluorescence excitation illumination source to accumulate charge at a plurality of pixels of the global shutter imager while the liquid crystal light shutter is in the closed state. The system reads a set of accumulated charge at the plurality of pixels of the global shutter imager to produce a set of imaging data and generates a fluorescence image frame based on the set of imaging data.

An exemplary method of imaging tissue of a subject using a fluorescence imaging system comprises a global shutter imager, a liquid crystal light shutter configurable to be in an open state and a closed state, and a fluorescence excitation illumination source. The method comprises: transitioning the liquid crystal light shutter to the closed state to prevent the global shutter imager from receiving visible light; illuminating the tissue of the subject with the fluorescence excitation illumination source to accumulate charge at a plurality of pixels of the global shutter imager, while the liquid crystal light shutter is in the closed state; reading a set of accumulated charge at the plurality of pixels of the global shutter imager to produce a set of imaging data; and generating a fluorescence image frame based on the set of imaging data.

In some examples, the method further comprises displaying the generated fluorescence image by adding the fluorescence image frame to a video stream.

In some examples, the fluorescence imaging system further comprises a visible-light illumination source, the set of accumulated charge is a first set of accumulated charge, and the set of imaging data is a first set of imaging data. The method further comprises: transitioning the liquid crystal light shutter to the open state to allow reflected light from illumination of the tissue of the subject with the visible-light illumination source to accumulate charge at the pixels of the global shutter imager; reading a second set of accumulated charge at the plurality of pixels of the global shutter imager to produce a second set of imaging data; and generating a visible-light image frame based on the second set of imaging data. In some examples, the fluorescence excitation illumination source is off during the illumination of the tissue of the subject with the visible-light illumination source. In some examples, the method further comprises: generating a blended image frame based on the fluorescence image frame and the visible-light image frame. In some examples, the fluorescence image frame is overlaid on the visible-light image frame in the blended image frame. In some examples, the blended image frame is derived from colorizing the visible-light image frame based on the fluorescence image frame. In some examples, the blended image frame is derived from colorizing the visible-light image frame based on the ratio of the fluorescence image frame to one or more channels of the visible-light frame.

In some examples, the method further comprises displaying the blended image frame by adding the blended image frame to a video stream.

In some examples, the visible-light illumination source is pulsed. The pulsed visible-light illumination source is configured to include: a plurality of primary visible-light illumination pulses for illuminating the tissue of the subject while the liquid crystal light shutter is in the open state; and one or more compensating visible-light illumination pulses between each two neighboring primary visible-light illumination pulses. In some examples, the liquid crystal light shutter is in the closed state during the one or more compensating visible-light illumination pulses. In some examples, the method further comprises while the fluorescence excitation illumination source is off and the liquid crystal light shutter is in the closed state, illuminating the tissue of the subject with ambient illumination to accumulate charge at the plurality of pixels of the global shutter imager; reading a third set of accumulated charge at the plurality of pixels of the global shutter imager to produce a third set of imaging data; and generating an ambient image frame based on the third set of imaging data. In some examples, the ambient illumination comprises ambient illumination in the fluorescence emission band. In some examples, the method further comprises: subtracting the ambient image frame from the fluorescence image frame.

In some examples, the visible-light illumination source is continuous. In some examples, the method further comprises: while the fluorescence excitation illumination source is off and the liquid crystal light shutter is in the closed state, illuminating the tissue of the subject with ambient illumination to accumulate charge at the plurality of pixels of the global shutter imager; reading a third set of accumulated charge at the plurality of pixels of the global shutter imager to produce a third set of imaging data; and generating an ambient image frame based on the third set of imaging data. The ambient illumination can comprise ambient illumination in the fluorescence emission band. In some examples, the method further comprises subtracting the ambient image frame from the fluorescence image frame.

An exemplary system of imaging tissue of a subject comprises: a fluorescence excitation illumination source, a visible-light illumination source, a liquid crystal light shutter configurable to be in an open state and a closed state, and an imager being configured for: transitioning the liquid crystal light shutter to the closed state to prevent the global shutter imager from receiving visible light; illuminating the tissue of the subject with the fluorescence excitation illumination source to accumulate charge at a plurality of pixels of the global shutter imager, while the liquid crystal light shutter is in the closed state; reading a set of accumulated charge at the plurality of pixels of the global shutter imager to produce a set of imaging data; and generating a fluorescence image frame based on the set of imaging data.

In some examples, the imager is further configured for: displaying the generated fluorescence image by adding the fluorescence image frame to a video stream.

In some examples, the fluorescence imaging system further comprises a visible-light illumination source, the set of accumulated charge is a first set of accumulated charge, and the set of imaging data is a first set of imaging data. The imager is further configured for: transitioning the liquid crystal light shutter to the open state to allow reflected light from illumination of the tissue of the subject with the visible-light illumination source to accumulate charge at the pixels of the global shutter imager; reading a second set of accumulated charge at the plurality of pixels of the global shutter imager to produce a second set of imaging data; and generating a visible-light image frame based on the second set of imaging data.

In some examples, the fluorescence excitation illumination source is off during the illumination of the tissue of the subject with the visible-light illumination source.

In some examples, the imager is further configured for: generating a blended image frame based on the fluorescence image frame and the visible-light image frame.

In some examples, the fluorescence image frame is overlaid on the visible-light image frame in the blended image frame. In some examples, the blended image frame is derived from colorizing the visible-light image frame based on the fluorescence image frame. In some examples, the blended image frame is derived from colorizing the visible-light image frame based on the ratio of the fluorescence image frame to one or more channels of the visible-light frame.

In some examples, the imager is further configured for: displaying the blended image frame by adding the blended image frame to a video stream.

In some examples, the visible-light illumination source is pulsed. The pulsed visible-light illumination source can be configured to include: a plurality of primary visible-light illumination pulses for illuminating the tissue of the subject while the liquid crystal light shutter is in the open state; and one or more compensating visible-light illumination pulses between each two neighboring primary visible-light illumination pulses.

In some examples, the liquid crystal light shutter is in the closed state during the one or more compensating visible-light illumination pulses.

In some examples, the imager is further configured for: while the fluorescence excitation illumination source is off and the liquid crystal light shutter is in the closed state, illuminating the tissue of the subject with ambient illumination to accumulate charge at the plurality of pixels of the global shutter imager; reading a third set of accumulated charge at the plurality of pixels of the global shutter imager to produce a third set of imaging data; and generating an ambient image frame based on the third set of imaging data. In some examples, the ambient illumination comprises light in the fluorescence emission band. In some examples, the imager is further configured for: subtracting the ambient image frame from the fluorescence image frame.

In some examples, the visible-light illumination source is continuous. The imager can be further configured for: while the fluorescence excitation illumination source is off and the liquid crystal light shutter is in the closed state, illuminating the tissue of the subject with ambient illumination to accumulate charge at the plurality of pixels of the global shutter imager; reading a third set of accumulated charge at the plurality of pixels of the global shutter imager to produce a third set of imaging data; and generating an ambient image frame based on the third set of imaging data.

In some examples, the ambient illumination comprises ambient light in the fluorescence emission band. In some examples, the imager is further configured for: subtracting the ambient image frame from the fluorescence image frame.

### BRIEF DESCRIPTION OF THE FIGURES

The invention will now be described, by way of example only, with reference to the accompanying drawings, in which:

FIG. 1A is an illustration of an endoscopic camera system, according to some examples;

FIG. 1B is a diagram of a portion of the endoscopic camera system of FIG. 1A and a target object for imaging, according to some examples;

FIG. 2A illustrates a schematic view of a system for illumination and imaging in open-field surgeries, according to some examples;

FIG. 2B illustrates another exemplary system for illumination and imaging in open-field surgeries, according to some examples;

FIG. 3 is a block diagram of an imaging system, according to some examples;

FIG. 4 provides an exemplary method for imaging tissue of a subject, in accordance with some examples;

FIG. 5A illustrates exemplary operations of an exemplary imaging system, in accordance with some examples;

FIG. 5B illustrates a timing diagram of the exemplary imaging system, in accordance with some examples;

FIG. 6A illustrates exemplary operations of an exemplary imaging system, in accordance with some examples;

FIG. 6B illustrates a timing diagram of the exemplary imaging system, in accordance with some examples;

FIG. 7A illustrates exemplary operations of an exemplary imaging system, in accordance with some examples;

FIG. 7B illustrates a timing diagram of the exemplary imaging system, in accordance with some examples;

FIG. 8A illustrates exemplary operations of an exemplary imaging system, in accordance with some examples; and

FIG. 8B illustrates a timing diagram of the exemplary imaging system, in accordance with some examples.

FIG. 9A illustrates an exemplary location of the liquid crystal light shutter, in accordance with some examples.

FIG. 9B illustrates an exemplary location of the liquid crystal light shutter, in accordance with some examples.

FIG. 9C illustrates an exemplary location of the liquid crystal light shutter, in accordance with some examples.

### DETAILED DESCRIPTION

Reference will now be made in detail to implementations and examples of various aspects and variations of systems and methods described herein. Although several exemplary variations of the systems and methods are described herein, other variations of the systems and methods may include aspects of the systems and methods described herein combined in any suitable manner having combinations of all or some of the aspects described. Examples will now be described more fully hereinafter with reference to the accompanying drawings; however, they may be embodied in different forms and should not be construed as limited to the examples set forth herein. Rather, these examples are provided so that this disclosure will be thorough and complete, and will fully convey exemplary implementations to those skilled in the art.

Examples of the present disclosure include various examples of an illumination scheme that acquire both fluorescence imaging data and corresponding visible-light imaging data using global shutter imagers (e.g., for surgical imaging applications). The techniques described herein can provide an improved medical imaging system that can alternate between acquisition of clean fluorescence imaging data (e.g., uncontaminated from ambient visible light) and acquisition of visible-light imaging data. In particular, the clean fluorescence imaging data is acquired via the use of a light shutter to block visible light. Examples of the present disclosure can either provide continuous visible-light illumination or visible-light illumination pulses to avoid strobe effects and jerky motion. The systems, devices, and methods described herein may be used for imaging tissue of a subject, such as in endoscopic imaging procedures and in open-field surgical procedures. Imaging may be performed pre-operatively, intra-operatively, post-operatively, and during diagnostic imaging sessions and procedures. In endoscopic imaging procedures, any imaging may be performed after the endoscope has been pre-inserted into a cavity. As such, any imaging method as disclosed herein may not include a step of inserting an endoscope into a cavity. Further, the techniques can be applied in non-surgical or non-medical uses.

The techniques described herein can utilize a light shutter, such as a liquid crystal (LC) shutter, for selective blocking of visible light from reaching the imager. The light shutter has an open state and a closed state and can transition between the two states based on a control signal for operating the light shutter, as described herein. In some examples, the light shutter is a liquid crystal (LC) shutter. The light shutter can behave differently for a light reflected from illumination of a tissue of the subject by the visible-light illumination source and a light emitted due to illumination of a tissue of the subject by the fluorescence excitation illumination source. Specifically, the light shutter can, in the open state, allow the passage of the light reflected from illumination of the tissue of the subject with the visible-light illumination such that the reflected light can reach the imager. In the closed state, the light shutter can prevent the light reflected from illumination of the tissue of the subject from reaching the imager or significantly attenuate the reflected light reaching the imager.

Further, regardless of which state the light shutter is in (e.g., the open state, the closed state, the transitioning state from the open state to the closed state, the transitioning state from the closed state to the open state), the light shutter can always allow the passage of a light emitted due to illumination of the tissue of the subject with the fluorescence excitation illumination source. For example, the light shutter may transmit light in the near infrared (NIR) range in both open and closed states, which can be useful for imaging compounds that fluoresce in the NIR. In other words, the light shutter can transmit the fluorescence light emitted due to illumination of the tissue of the subject with the fluorescence excitation illumination even when the light shutter is in the closed state, so the opening and/or closing of the light shutter only affects the passage of the light reflected from illumination of the tissue of the subject with the visible-light illumination. When the light shutter is in the closed state, no light reflected from illumination of the tissue of the subject with the visible-light illumination source is transmitted; instead, only the light emitted due to illumination of the tissue of the subject with the fluorescence excitation illumination source is transmitted.

In some examples of an illumination scheme, an exemplary system comprises a global shutter imager, a liquid crystal light shutter configurable to be in an open state and a closed state, and a fluorescence excitation illumination source. The system transitions the liquid crystal light shutter to the closed state to prevent the global shutter imager from receiving visible light and illuminates the tissue of the subject with the fluorescence excitation illumination source to accumulate charge at a plurality of pixels of the global shutter imager, while the liquid crystal light shutter is in the closed state. The system reads a set of accumulated charge at the plurality of pixels of the global shutter imager to produce a set of imaging data and generates a fluorescence image frame based on the set of imaging data.

In the following description, it is to be understood that the singular forms “a,” “an,” and “the” used in the following description are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

Certain aspects of the present disclosure include process steps and instructions described herein in the form of an algorithm. It should be noted that the process steps and instructions of the present disclosure could be embodied in software, firmware, or hardware and, when embodied in software, could be downloaded to reside on and be operated from different platforms used by a variety of operating systems. Unless specifically stated otherwise as apparent from the following discussion, it is appreciated that, throughout the description, discussions utilizing terms such as “processing,” “computing,” “calculating,” “determining,” “displaying,” “generating” or the like, refer to the action and processes of a computer system, or similar electronic computing device, that manipulates and transforms data represented as physical (electronic) quantities within the computer system memories or registers or other such information storage, transmission, or display devices.

The present disclosure in some examples also relates to a device for performing the operations herein. This device may be specially constructed for the required purposes, or it may comprise a general purpose computer selectively activated or reconfigured by a computer program stored in the computer. Such a computer program may be stored in a non-transitory, computer readable storage medium, such as, but not limited to, any type of disk, including floppy disks, USB flash drives, external hard drives, optical disks, CD-ROMs, magnetic-optical disks, read-only memories (ROMs), random access memories (RAMs), EPROMS, EEPROMs, magnetic or optical cards, application specific integrated circuits (ASICs), or any type of media suitable for storing electronic instructions, and each coupled to a computer system bus. Furthermore, the computers referred to in the specification may include a single processor or may be architectures employing multiple processor designs for increased computing capability.

The methods, devices, and systems described herein are not inherently related to any particular computer or other apparatus. Various general-purpose systems may also be used with programs in accordance with the teachings herein, or it may prove convenient to construct a more specialized apparatus to perform the required method steps. The required structure for a variety of these systems will appear from the description below. In addition, the present invention is not described with reference to any particular programming language. It will be appreciated that a variety of programming languages may be used to implement the teachings of the present invention as described herein.

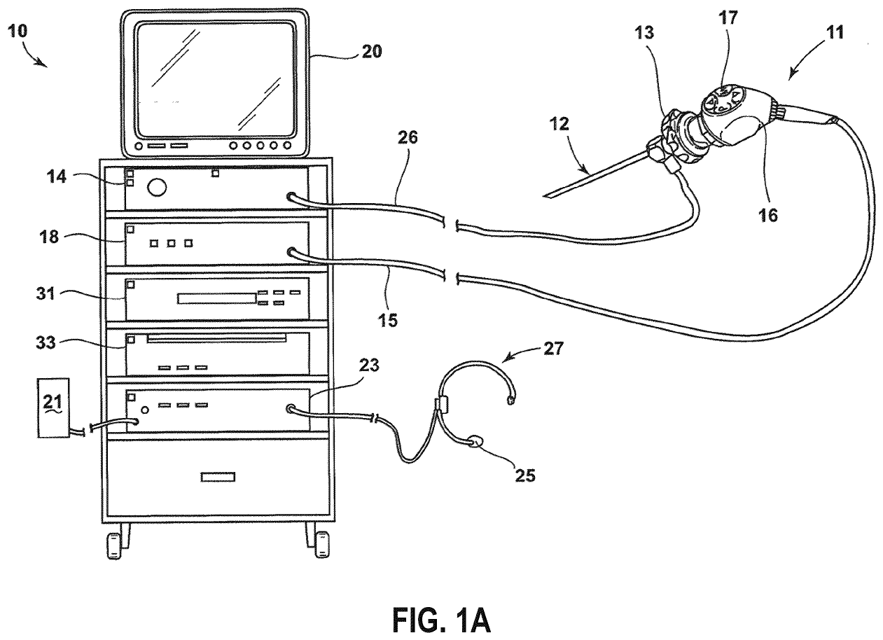

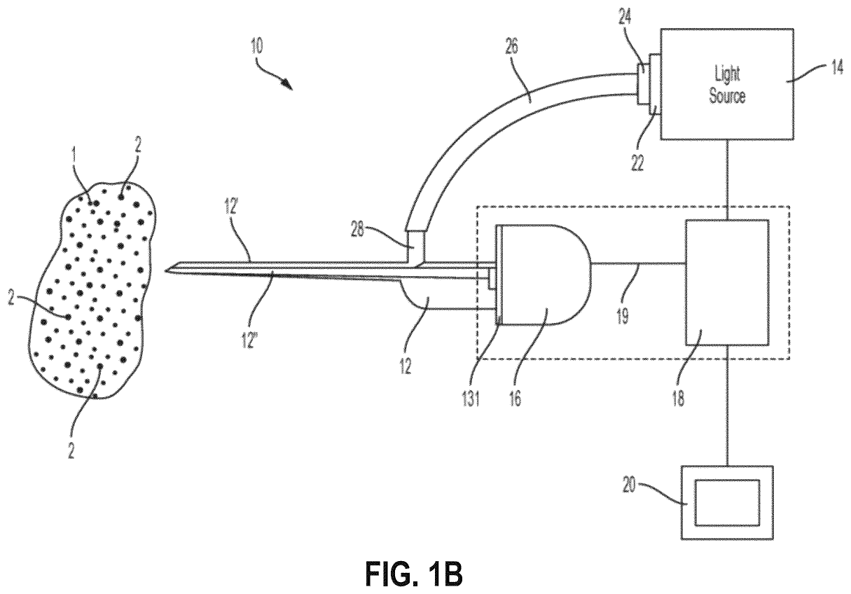

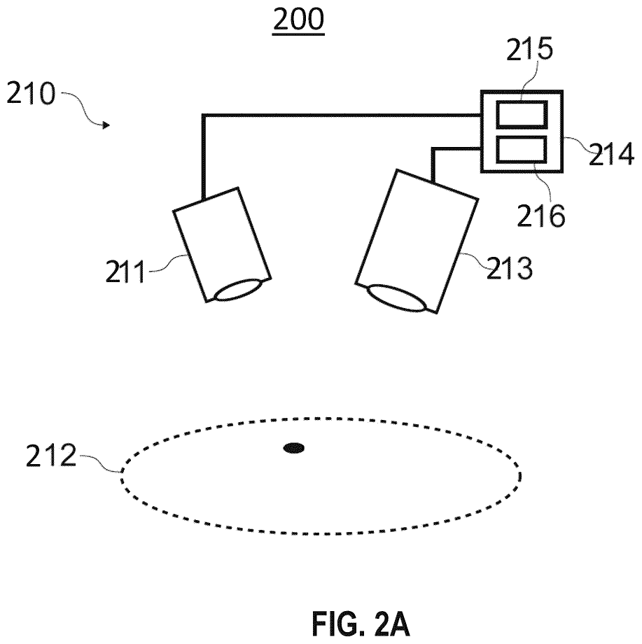

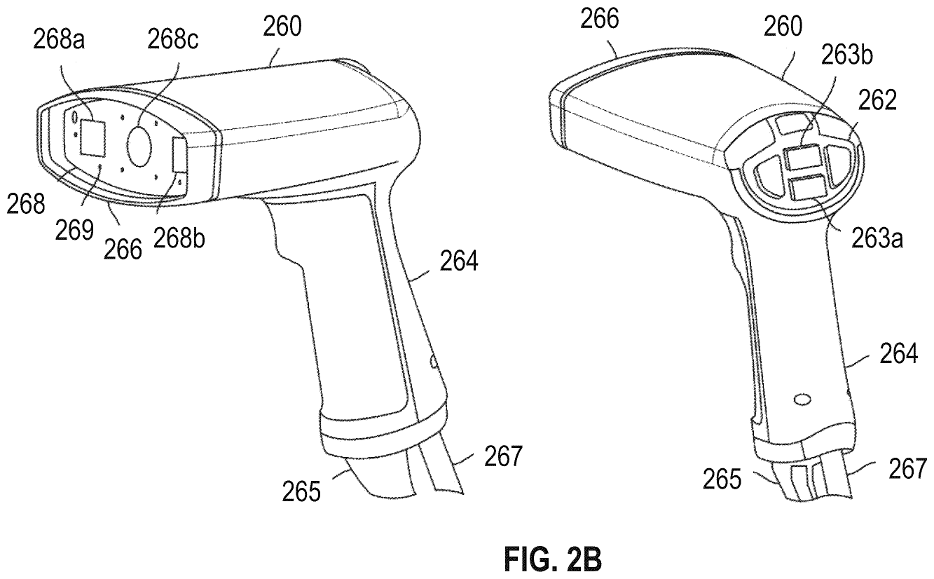

Examples of the present disclosure may be incorporated into any medical imaging systems, including imaging systems used in minimally invasive surgeries (e.g., FIGS. 1A-B) and open field imaging systems (e.g., FIG. 2A). FIG. 1A shows an example of an endoscopic imaging system 10, which includes a scope assembly 11 which may be utilized in endoscopic procedures. The scope assembly 11 incorporates an endoscope or scope 12 which is coupled to a camera head 16 by a coupler 13 located at the distal end of the camera head 16. Light is provided to the scope by a light source 14 via a light guide 26, such as a fiber optic cable. The camera head 16 is coupled to a camera control unit (CCU) 18 by an electrical cable 15. The CCU 18 is connected to, and communicates with, the light source 14. Operation of the camera 16 is controlled, in part, by the CCU 18. The cable 15 conveys video image and/or still image data from the camera head 16 to the CCU 18 and may convey various control signals bi-directionally between the camera head 16 and the CCU 18.

A control or switch arrangement 17 may be provided on the camera head 16 for allowing a user to manually control various functions of the system 10, which may include switching from one imaging mode to another, as discussed further below. Voice commands may be input into a microphone 25 mounted on a headset 27 worn by the practitioner and coupled to the voice-control unit 23. A hand-held control device 29, such as a tablet with a touch screen user interface or a PDA, may be coupled to the voice control unit 23 as a further control interface. In the illustrated example, a recorder 31 and a printer 33 are also coupled to the CCU 18. Additional devices, such as an image capture and archiving device, may be included in the system 10 and coupled to the CCU 18. Video image data acquired by the camera head 16 and processed by the CCU 18 is converted to images, which can be displayed on a monitor 20, recorded by recorder 31, and/or used to generate static images, hard copies of which can be produced by the printer 33.

FIG. 1B shows an example of a portion of the endoscopic system 10 being used to illuminate and receive light from an object 1, such as a surgical site of a patient. The object 1 may include fluorescent markers 2, for example, as a result of the patient being administered a fluorescence imaging agent. The fluorescent markers 2 may comprise, for example, indocyanine green (ICG).

The light source 14 can generate visible illumination light (such as any combination of red, green, and blue light) for generating visible (e.g., white light) images of the target object 1 and, in some examples, can also produce fluorescence excitation illumination light for exciting the fluorescent markers 2 in the target object for generating fluorescence images. In some examples, the light source 14 can produce fluorescence excitation illumination light for exciting autofluorescence in the target object for generating fluorescence images, additionally or alternatively to light for exciting the fluorescent markers. Illumination light is transmitted to and through an optic lens system 22 which focuses light onto a light pipe 24. The light pipe 24 may create a homogeneous light, which is then transmitted to the fiber optic light guide 26. The light guide 26 may include multiple optic fibers and is connected to a light post 28, which is part of the endoscope 12. The endoscope 12 includes an illumination pathway 12′ and an optical channel pathway 12″.

The endoscope 12 may include a notch filter 131 that allows some or all (preferably, at least 80%) of fluorescence emission light (e.g., in a wavelength range of 830 nm to 870 nm) emitted by fluorescence markers 2 in the target object 1 to pass therethrough and that allows some or all (preferably, at least 80%) of visible light (e.g., in the wavelength range of 400 nm to 700 nm), such as visible illumination light reflected by the target object 1, to pass therethrough, but that blocks substantially all of the fluorescence excitation light (e.g., infrared light having an infrared wavelength such as 780 nm, 808 nm, or the like) that is used to excite fluorescence emission from the fluorescent marker 2 in the target object 1. The notch filter 131 may have an optical density of OD5 or higher. In some examples, the notch filter 131 can be located in the coupler 13.

FIG. 2A illustrates an exemplary open field imaging system in accordance with some examples. FIG. 2A illustrates a schematic view of an illumination and imaging system 210 that can be used in open field surgical procedures. As may be seen therein, the system 210 may include an illumination module 211, an imaging module 213, and a video processor/illuminator (VPI) 214. The VPI 214 may include an illumination source 215 to provide illumination to the illumination module 211 and a processor assembly 216 to send control signals and to receive data about light detected by the imaging module 213 from a target 212 illuminated by light output by the illumination module 211. In one variation, the video processor/illuminator 214 may comprise a separately housed illumination source 215 and the processor assembly 216. In one variation, the video processor/illuminator 214 may comprise the processor assembly 216 while one or more illumination sources 215 are separately contained within the housing of the illumination module 211. The illumination source 215 may output light at different waveband regions, e.g., white (RGB) light, excitation light to induce fluorescence in the target 212, a combination thereof, and so forth, depending on characteristics to be examined and the material of the target 212. Light at different wavebands may be output by the illumination source 215 simultaneously, sequentially, or both. The illumination and imaging system 210 may be used, for example, to facilitate medical (e.g., surgical) decision-making, e.g., during a surgical procedure. The target 212 may include fluorescent markers, for example, as a result of the patient being administered a fluorescence imaging agent. The fluorescent markers may comprise, for example, indocyanine green (ICG). The target 212 may be a topographically complex target, e.g., a biological material including tissue, an anatomical structure, other objects with contours and shapes resulting in shadowing when illuminated, and so forth. The VPI 214 may record, process, display, and so forth, the resulting images and associated information.

FIG. 2B illustrates an additional example of an open field surgical imaging system or a portion thereof. With reference to FIG. 2B, an ergonomic enclosure 260 can be designed to be held in a pistol-style grip. The enclosure 260 may include a control surface 262, a grip 264, a window frame 268 and a nosepiece 266. The ergonomic enclosure 260 is connectable to a VPI box (not depicted) via a light guide cable 267, through which the light is provided to one or more illumination ports (not depicted), and a data cable 265 that transmits power, sensor data, and any other (non-light) connections.

The control surface 262 includes focus buttons 263a and 263b that control the focus actuation assembly. Other buttons on the control surface 262 may be programmable and may be used for various other functions, excitation laser power on/off, display mode selection, white light imaging white balance, saving a screenshot, and so forth. Alternatively or additionally to the focus buttons, a proximity sensor may be provided on the enclosure and may be employed to automatically adjust the focus actuation assembly.

Enclosure 260 may be operated by a single hand in a pistol-grip style orientation. In various other examples, the enclosure 260 may be supported on a support (e.g., a movable support). In some examples, enclosure 260 may be used in concert with a drape. Window frame 268 may include windows 268a and 268b, corresponding to two lens modules, as well as window 268c, which serves as an input window for light from the target to be incident on the image sensor. Window frame 268 may also include one or more windows 269 for sensors provided behind a plate.

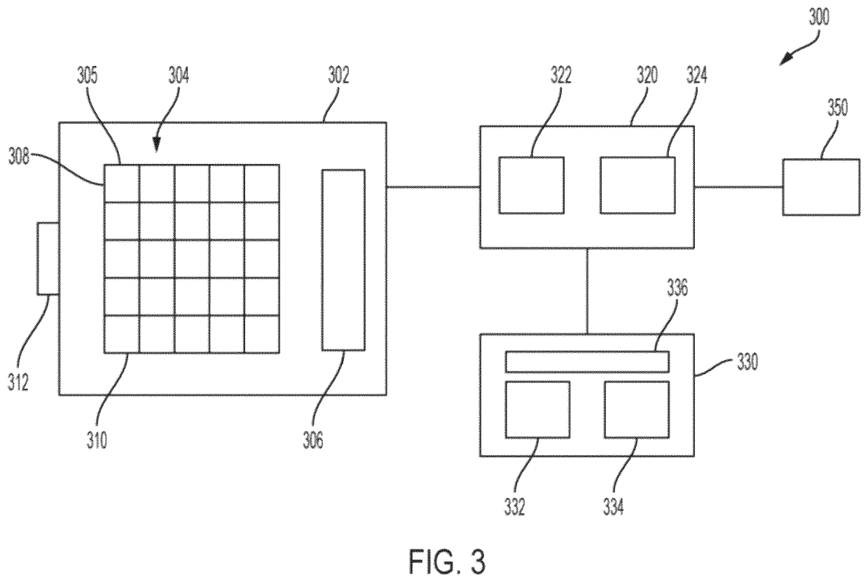

FIG. 3 schematically illustrates an exemplary imaging system 300 that employs an electronic imager 302 to generate images (e.g., still and/or video) of a target object, such as a target tissue of a patient, according to some examples. The imager 302 may be a global shutter imager (e.g., CCD sensors, CMOS sensors). System 300 may be used, for example, for the endoscopic imaging system 10 of FIG. 1A. The imager 302 includes a CMOS sensor 304 having an array of pixels 305 arranged in rows of pixels 308 and columns of pixels 310. The imager 302 may include control components 306 that control the signals generated by the CMOS sensor 304. Examples of control components include gain circuitry for generating a multi-bit signal indicative of light incident on each pixel of the sensor 304, one or more analog-to-digital converters, one or more line drivers to act as a buffer and provide driving power for the sensor 304, row circuitry, and timing circuitry. A timing circuit may include components such as a bias circuit, a clock/timing generation circuit, and/or an oscillator. Row circuitry may enable one or more processing and/or operational tasks such as addressing rows of pixels 308, addressing columns of pixels 310, resetting charge on rows of pixels 308, enabling exposure of pixels 305, decoding signals, amplifying signals, analog-to-digital signal conversion, applying timing, readout, and reset signals, and other suitable processes or tasks. Imager 302 may also include a shutter 312 that may be used, for example, to control exposure of the image sensor 304 and/or to control an amount of light received at the image sensor 304.

One or more control components may be integrated into the same integrated circuit in which the sensor 304 is integrated or may be discrete components. The imager 302 may be incorporated into an imaging head, such as camera head 16 of system 10.

One or more control components 306, such as row circuitry and a timing circuit, may be electrically connected to an imaging controller 320, such as camera control unit 18 of system 10. The imaging controller 320 may include one or more processors 322 and memory 324. The imaging controller 320 receives imager row readouts and may control readout timings and other imager operations, including mechanical shutter operation. The imaging controller 320 may generate image frames, such as video frames from the row and/or column readouts from the imager 302. Generated frames may be provided to a display 350 for display to a user, such as a surgeon.

The system 300 in this example includes a light source 330 for illuminating a target scene. The light source 330 is controlled by the imaging controller 320. The imaging controller 320 may determine the type of illumination provided by the light source 330 (e.g., white light, fluorescence excitation light, or both), the intensity of the illumination provided by the light source 330, and or the on/off times of illumination in synchronization with image sensor shutter operation. The light source 330 may include a first light generator 332 for generating light in a first wavelength and a second light generator 334 for generating light in a second wavelength. In some examples, the first light generator 332 is a white light generator, which may be comprised of multiple discrete light generation components (e.g., multiple LEDs of different colors), and the second light generator 334 is a fluorescence excitation light generator, such as a laser diode.

The light source 330 includes a controller 336 for controlling light output of the light generators. The controller 336 may be configured to provide pulse width modulation (PWM) of the light generators for modulating intensity of light provided by the light source 330, which can be used to manage overexposure and underexposure. In some examples, nominal current and/or voltage of each light generator remains constant, and the light intensity is modulated by switching the light generators (e.g., LEDs) on and off according to a PWM control signal. In some examples, a PWM control signal is provided by the imaging controller 336. This control signal can be a waveform that corresponds to the desired pulse width modulated operation of light generators.

The imaging controller 320 may be configured to determine the illumination intensity required of the light source 330 and may generate a PWM signal that is communicated to the light source 330. In some examples, depending on the amount of light received at the sensor 304 and the integration times, the light source may be pulsed at different rates to alter the intensity of illumination light at the target scene. The imaging controller 320 may determine a required illumination light intensity for a subsequent frame based on an amount of light received at the sensor 304 in a current frame and/or one or more previous frames. In some examples, the imaging controller 320 is capable of controlling pixel intensities via PWM of the light source 330 (to increase/decrease the amount of light at the pixels), via operation of the shutter 312 (to increase/decrease the amount of light at the pixels), and/or via changes in gain (to increase/decrease sensitivity of the pixels to received light). In some examples, the imaging controller 320 primarily uses PWM of the illumination source for controlling pixel intensities while holding the shutter open (or at least not operating the shutter) and maintaining gain levels. The controller 320 may operate the shutter 312 and/or modify the gain in the event that the light intensity is at a maximum or minimum and further adjustment is needed.

### Method for Imaging Tissue of a Subject

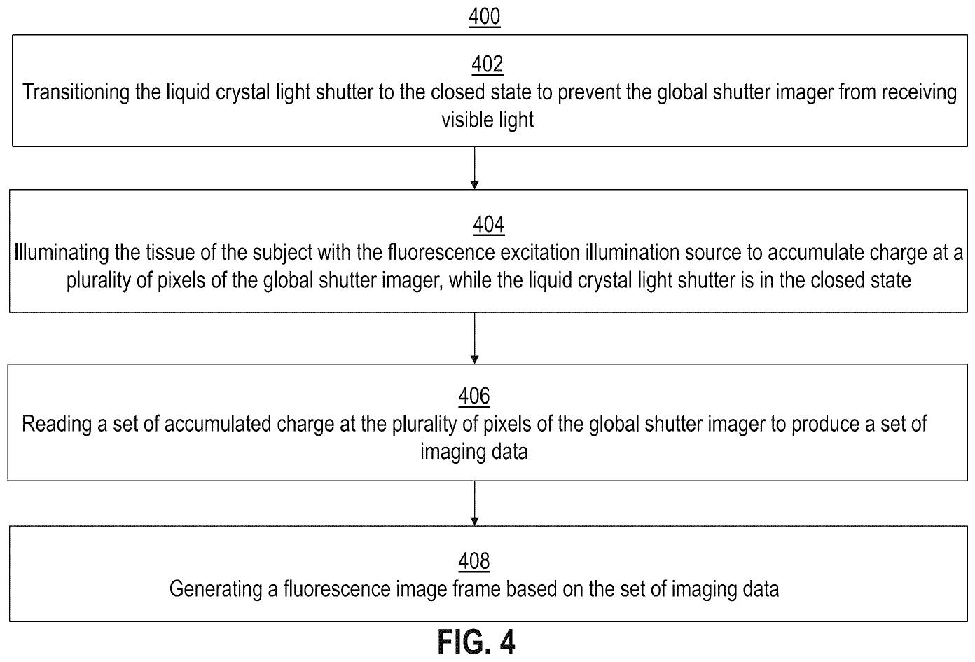

FIG. 4 provides an exemplary method 400 for imaging tissue of a subject, in accordance with some examples. The exemplary imaging system can include a global shutter imager, a fluorescence excitation illumination source, a visible-light illumination source, and a light shutter. The imaging system can illuminate the tissue of the subject using a combination of the fluorescence excitation illumination source and the visible-light illumination source to accumulate charges at a plurality of rows of pixels of the global shutter imager, as described herein. In some examples, the imager is part of an endoscopic imager or an open-field imager and may comprise a CMOS sensor. In some examples, the imaging system is the imaging system 300 of FIG. 3, which has an imager (e.g., global shutter imager 302 of system 300) and a light source (e.g., light source 330 of system 300) that can comprise a fluorescence excitation illumination source and/or a visible-light illumination source.

The fluorescence excitation illumination source can be configured to provide fluorescence excitation illumination (e.g., infrared light) to the tissue to be imaged. The fluorescence excitation illumination can excite fluorescent markers in the tissue to emit light that in turn can reach the global shutter imager to generate fluorescence imaging data. In some examples, the fluorescence excitation illumination source can comprise a laser diode, such as a near-infrared (NIR) excitation light.

The visible-light illumination source can generate visible illumination light (such as any combination of red, green, and blue light) to illuminate the tissue to generate reflected light that in turn can reach the global shutter imager to generate visible-light imaging data. In some examples, the visible-light illumination source can comprise multiple discrete light generation components (e.g., multiple LEDs of different colors). In some examples, the visible-light illumination source can comprise a RGB light, such as a pulsing LED.

A global shutter comprises a sensor and allows all pixels in the sensor to be exposed simultaneously, thus capturing the entire image at once. Specifically, all pixels in the sensor are exposed to illumination simultaneously for the duration of the exposure time. During the exposure period, each pixel accumulates charge. Once the exposure period is complete, at the start of the readout time, the charges accumulated in all pixels are transferred to holding capacitors simultaneously, and then read out (e.g., converted into a digital value or voltage value) and transferred to the memory for further processing. The accumulated charges in all pixels can be reset. This process is opposed to a rolling shutter, where the exposure occurs sequentially, line by line, which may lead to distortions and artifacts (e.g., in moving objects, when the camera is in motion).

In some examples, the global shutter imager can include a complimentary metal-oxide-semiconductor (CMOS) sensor having an array of pixels arranged in rows of pixels and columns of pixels. In some examples, the global shutter imager can output image frames at the CMOS sensor's frame rate (e.g., 120 Hz), which can be used to generate an image display output. The global shutter imager can include a mechanical shutter that can be used, for example, to control exposure of the CMOS sensor and/or to control an amount of light received at the CMOS sensor. In some examples, the global shutter imager can include a RGB prism camera with global shutter sensors. In some examples, the global shutter imager can include a RGB prism camera with one or more global shutter sensors, or a single-sensor camera with a Bayer-filter global shutter sensor (e.g., running at 120 fps or faster).

The light shutter has an open state and a closed state and can transition between the two states based on a control signal for operating the light shutter, as described herein. In some examples, the light shutter is an electronically formed shutter. In some examples, the light shutter is a liquid crystal (LC) shutter. The light shutter is configurable to switch between the open state and the closed state in a relatively short period of time comparing to the frame rate of the imaging system. In some examples, the time for the light shutter to transition from one state to the other state can be lower than 17 milliseconds. In some examples, the time for the light shutter to transition from one state to the other state can be lower than 10 milliseconds. It should be appreciated by one of ordinary skill in the art that the time to transition from the open state to the closed state may be differ from the time to transition from the closed state to the open state due to the nature of the liquid crystal material. In some examples, the light shutter is configurable to switch from the closed state to the open state in less than 1.8 milliseconds and transition from the open state to the closed state in less than 100 microseconds. In some examples, the light shutter can be configured to operate in accordance with reversed timing (e.g., switching from the closed state to the open state in less than 100 microseconds and transition from the open state to the closed state in less than 1.8 milliseconds). It should be appreciated that the ranges described herein are merely exemplary and are not intended to be limiting.

The light shutter may be placed in any location in front of the optical sensors of the global shutter imager of the imaging system. In some examples, the light shutter is placed behind a unit of one or more lens (hereinafter a “lens unit”) and in front of the optical sensors. In some examples, the light shutter is placed in front of the lens unit or within the lens unit. In some examples, the light shutter is placed in front of an optical prism of the imaging system. In some examples, the light shutter can comprise multiple sub-shutters (e.g., corresponding to multiple optical sensors of the global shutter imager).

In process 400, some blocks are optionally combined, the order of some blocks is optionally changed, and some blocks are optionally omitted. In some examples, additional steps may be performed in combination with the process 400. Accordingly, the operations as illustrated (and described in greater detail below) are exemplary by nature and, as such, should not be viewed as limiting.

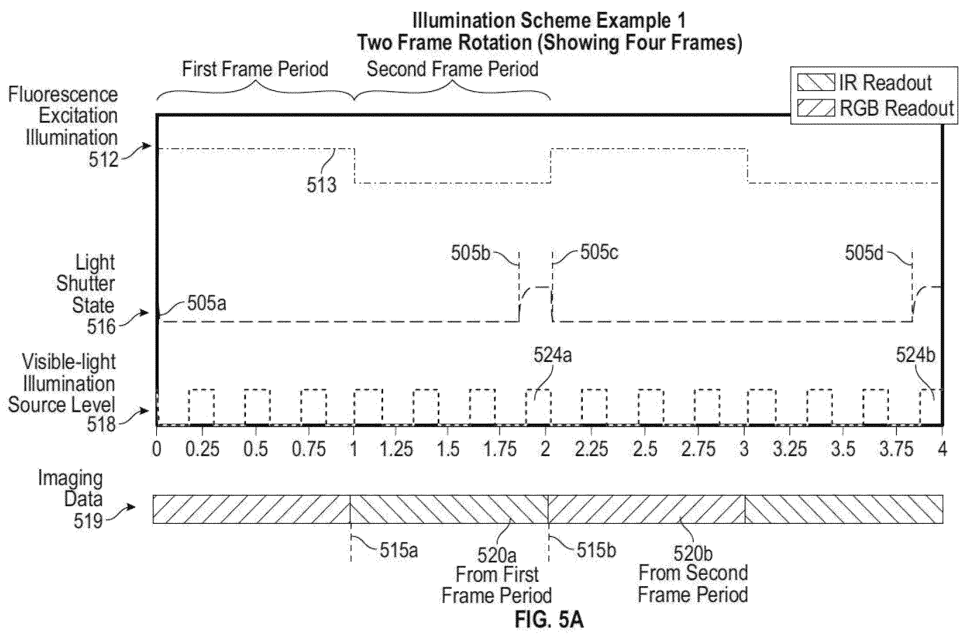

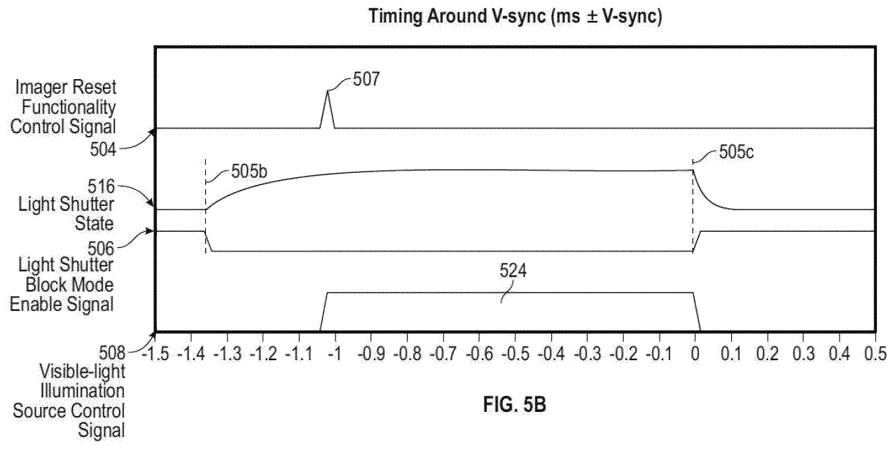

At block 402, the system transitions the liquid crystal light shutter to the closed state to prevent the global shutter imager from receiving visible light. At block 404, the system illuminates the tissue of the subject with the fluorescence excitation illumination source to accumulate charge at a plurality of pixels of the global shutter imager, while the liquid crystal light shutter is in the closed state. At block 406, the system reads a set of accumulated charge at the plurality of pixels of the global shutter imager to produce a set of imaging data. At block 408, the system generates a fluorescence image frame based on the set of imaging data. Examples of the method 400 are provided herein with reference to FIGS. 5A-8B.

### Example One of the Illumination Scheme

FIGS. 5A and 5B illustrate timing diagrams for operating an imaging system for imaging tissue from a subject, in accordance with some examples. In FIG. 5A, the illumination scheme involves a series of two-image rotations. Each rotation produces two images: a fluorescence image corresponding to a first frame period and a visible-light image corresponding to a second frame period. In each of the timing diagrams described herein (FIGS. 5A-8B), the values on the x-axis indicate fractional or whole frame periods rather than absolute time units. Accordingly, the timing diagrams do not depict any particular frequencies. If the image sensor runs at 120 fps, this results in a 60 fps update of the output video, which is sufficient for continuous-appearing video without noticeable jerkiness.

With reference to FIG. 5A, the illumination level 512 of the fluorescence excitation illumination source is shown to comprise a sequence of fluorescence excitation illumination pulses, indicating that the fluorescence excitation illumination source is configured to provide fluorescence excitation illumination periodically for the duration of the pulse width shown in the illumination level 512. As one example of the illumination scheme, the frequency of the sequence of fluorescence illumination pulses can be 60 Hz, turning on approximately at a vertical synchronization event (V-sync) where the primary visible-light illumination pulse ends, and off approximately at the next V-sync. Further, the illumination level 518 of the visible-light illumination source is also shown to comprise a sequence of visible-light illumination pulses, indicating that the visible-light illumination source is configured to provide visible-light illumination periodically for the duration of the pulse width shown in the illumination level 518. As one example of the illumination scheme, the frequency of the sequence of visible-light illumination pulses can be 420 Hz. The light shutter state 516 can be open (shown as high) or closed (shown as low). As shown in FIG. 5A, the light shutter is generally in the closed state but is transitioned into the open state periodically.

At the beginning of the first frame period, at time 505a, the light shutter transitions from the open state to the closed state to prevent the global shutter imager from receiving any visible light. Specifically, the light shutter prevents the passage of light reflected from illumination of the tissue of the subject with any visible-light illumination pulse. Further, while the light shutter is in the closed state, the light shutter still allows the passage of the light emitted due to illumination of the tissue of the subject with the fluorescence excitation illumination source to reach the global shutter imager. Thus, during the first frame period, the fluorescence excitation illumination source can illuminate the tissue of the subject with the fluorescence excitation illumination pulse 513 to accumulate charge at a plurality of pixels of the global shutter imager, while the liquid crystal light shutter is in the closed state.

At the end of the first frame period (i.e., at readout time 515a), the imaging system begins reading out a first set of accumulated charge at the plurality of pixels of the global shutter imager to produce a first set of imaging data 520a. The first set of imaging data 520a contains only fluorescence imaging data because the light shutter prevented the global shutter imager from receiving any visible light during the first frame period. Accordingly, the imaging system generates a fluorescence image frame based on the first set of imaging data 520a. When readout begins at the readout time 515a, the charges have been transferred to holding capacitors and the pixels all reset, so the imager can begin accumulating the next image, and the imager may optionally be controlled to reset the charge part way through that period in order to adjust the exposure time (not depicted).

During the second frame period, at time 505b, the light shutter transitions from the closed state to the open state to allow reflected light from illumination of the tissue of the subject with the visible-light illumination source to accumulate charge at the pixels of the global shutter imager. In the depicted example, the light shutter transitions from the closed state to the open state at time 505b and then transitions from the open state back to the closed state at time 505c. While the light shutter is open, the reflected light from illumination of the tissue of the subject with the visible-light illumination pulse 524a to accumulate charge at the pixels of the global shutter imager. Further, during the illumination of the tissue of the subject with the visible-light illumination pulse 524a, the fluorescence excitation illumination source is off.

At the end of the second frame period (i.e., at the readout time 515b), the imaging system begins reading out a second set of accumulated charge at the plurality of pixels of the global shutter imager to produce a second set of imaging data 520b. The second set of imaging data 520b contains only visible-light imaging data because the light shutter was open to permit passage of visible light and the fluorescence excitation illumination source was off during the second frame period. Accordingly, the imaging system generates a visible-light image frame based on the second set of imaging data 520b.

Accordingly, the imaging system can generate a fluorescence image frame based on the first set of imaging data 520a and a visible-light image frame based on the second set of imaging data 520b. Another type of image frame can be a blended image frame based on both the fluorescence image frames and the visible-light image frame. In some examples, the fluorescence image frame can be overlaid on the visible-light image frame in the blended image frame. In some examples, the blended image frame can be derived from colorizing the visible-light image frame based on the fluorescence image frames (e.g., based on the ratio of the fluorescence image frame to one or more channels of the visible-light frame). One or more of the generated image frames can be added to a video stream and displayed on a display to a user (e.g., a medical practitioner such as a surgeon).

In the illumination scheme in FIG. 5A, the light shutter is configured to periodically open (e.g., at 505b, 505d) each time to allow the reflected light from illumination of the tissue of the subject with a single visible-light illumination pulse (e.g., 524a, 524b) to pass through the light shutter and reach the global shutter imager. These visible-light illumination pulses (e.g., 524a, 524b) are referred to as “primary” visible-light illumination pulses. Between two neighboring primary visible-light illumination pulses, there can be one or more “compensating” visible-light illumination pulses. In the depicted example, between two neighboring primary visible-light illumination pulses (e.g., 524a and 524b), there are six compensating visible-light illumination pulses. As shown in FIG. 5A, the light shutter is in a closed state during the compensating visible-light illumination pulses, thus preventing the reflected light from illumination of the tissue of the subject with the compensating visible-light illumination pulses from passing through the light shutter to reach the global shutter imager.

Although the compensation pulses are not utilized by the global shutter imager to generate imaging data, the presence of the compensating visible-light illumination pulses can improve a user's visual comfort when the imaging system is in operation. Without the compensating visible-light illumination pulses, the primary visible-light illumination pulses alone can occur at a frequency (e.g., 60 Hz) low enough to generate a visual strobe effect, which can be uncomfortable for the user (e.g., a medical practitioner such as a surgeon) and thus compromise patient safety. By increasing the number and frequency of pulses via added compensating visible-light illumination pulses, the visible-light illumination source can appear to generate a more continuous light, which can increase the user's visual comfort. For example, with a small number of compensating visible-light illumination pulses (e.g., 1 to 3 pulses), the pulses can occur at a frequency of 120 to 240 Hz. This produces visible-light illumination that appears continuous to the human eye, but causes a strobe effect on illuminated objects as they move across the field of illumination. The strobe effect decreases as the number of compensating visible-light illumination pulses increases and can be effectively imperceptible to the user with 6 compensating visible-light illumination pulses (e.g., the pulses occur at a frequency of 420 Hz). In some examples, the number of compensating visible-light illumination pulses can be as 2, 3, 4, or 5. The maximum number can be bounded by the off period needing to be longer than the ramp-open time of the LC shutter, and the on period needing to be long enough to acquire a reasonably bright image. The minimum number can be bounded to two (which is equivalent to 90 Hz) to ensure that the illumination looks continuous to the eye. In any of the examples described herein, the compensating visible-light illumination pulses preferably may have the same duration as the primary visible-light illumination pulses for the user's visual comfort.

FIG. 5B illustrates a timing diagram with a zoomed-in view of the time period around the end of the second frame period, in accordance with some examples. The x-axis of the timing diagram represents time in milliseconds relative to a vertical synchronization event (V-sync). FIG. 5B shows the alignment of a visible-light illumination pulse with the V-sync and the timing of the light shutter. Specifically, FIG. 5B illustrates control signals for operating various components of the imaging system, in accordance with some examples, including a control signal 504 for operating an optional reset functionality of the global shutter imager, a control signal 506 for operating the light shutter, and a control signal 508 for operating the visible-light illumination source.

With reference to FIG. 5B, the control signal 506 for operating the light shutter controls the timing for transitioning the light shutter between the closed state and the open state. In the depicted example, the control signal 506 dictates when to enable a “block mode (i.e., the closed state) of the light shutter. At any given time, the control signal 506 can be either high (indicating that the “block mode” is enabled) or low (indicating that the “block mode” is disabled). Further with reference to FIG. 5B, the control signal 504 controls the timing for activating an optional reset functionality of the global shutter imager. All rows of pixels in the image sensor can be reset simultaneously or within a short time period upon triggering the control signal 504 (e.g., in addition to being automatically reset with each readout).

As shown in FIG. 5B, at the time 505b, the control signal 506 for operating the light shutter changes from high (i.e., block mode enabled) to low (i.e., block mode disabled). In response to the control signal 506, the light shutter starts to transition from the closed state to the open state starting at time 505b, as shown by the gradual upward curvature of the light shutter state 516 starting from the time 505b. The gradual upward curvature of the light shutter state 516 on a millisecond time scale indicates that the light shutter does not instantly transition from the closed state to the open state. Rather, the transitioning is gradual over time and thus the amount of light passage gradually increases over time during the transitioning. It should be appreciated by one of ordinary skill in the art that the gradual nature of the transitioning is due to the nature of the liquid crystal material.

Then at the time 505c, the control signal 506 for operating the light shutter changes from low (i.e., block mode disabled) to high (i.e., block mode enabled). In response to the control signal 506, the light shutter starts to transition from the open state to the closed state starting at the time 505c. The rapid downward curvature of the light shutter state 516 indicates that light shutter does not instantly transition from the open state to the closed state. Rather, the transitioning is gradual over a very short time and thus the amount of light passage decreases over that time due to the nature of the liquid crystal material.

Notably in FIG. 5B, the visible-light illumination source is activated, by transitioning the visible-light illumination source control signal 524 from low to high, to provide a visible-light illumination pulse 524a after the time 505b. As shown in FIG. 5B, the transitioning of the light shutter from the closed state to the open state is complete or substantially complete when the visible-light illumination pulse 524a starts, thus allowing the passage of the light reflected from illumination of the tissue of the subject with the visible-light illumination pulse 524a to reach the global shutter imager. Further, the control signal 504 for operating the reset functionality triggers at time 507, resetting of all rows of pixels in the image sensor of the global shutter imager simultaneously or within a short time period. The control signal 504 for operating the reset functionality can occur anywhere between time 507 and V-sync to adjust the exposure period for the visible-light image and thus to control the brightness of the visible-light image. The closer the control signal is to the time 507, the brighter the visible-light image is. For example, in any of the examples disclosed herein, when the camera is closer to the subject, the imaging system may shift the timing of the reset pulse to reduce the brightness.

Further in FIG. 5B, the visible-light illumination source is deactivated to end the visible-light illumination pulse 524a at the same as the time 505c when the light shutter starts to transition to the closed state. This way, the transitioning of the light shutter to the closed state would not block the passage of the light reflected from illumination of the tissue of the subject with the visible-light illumination pulse 524a. Further, the transitioning of the light shutter from the open state to the closed state is complete before the next visible-light illumination pulse starts. As discussed herein, the next visible-light illumination pulse is a compensating pulse and is blocked by the light shutter from reaching the global shutter imager.

The example in FIGS. 5A-B provides several technical advantages. The two-frame rotation allows for continuous 60 Hz output with a 120 Hz image sensor. All of the fluorescence excitation illumination is used for imaging fluorescence, so there is no wasted fluorescence excitation illumination. Further, by pulsing the fluorescence excitation illumination source rather than running it continuously, there is no need to correct for fluorescence in the visible-light image frame. Further, the fluorescence imaging data is not contaminated by any visible light and thus there is no need to acquire ambient visible-light imaging data.

### Example Two of the Illumination Scheme

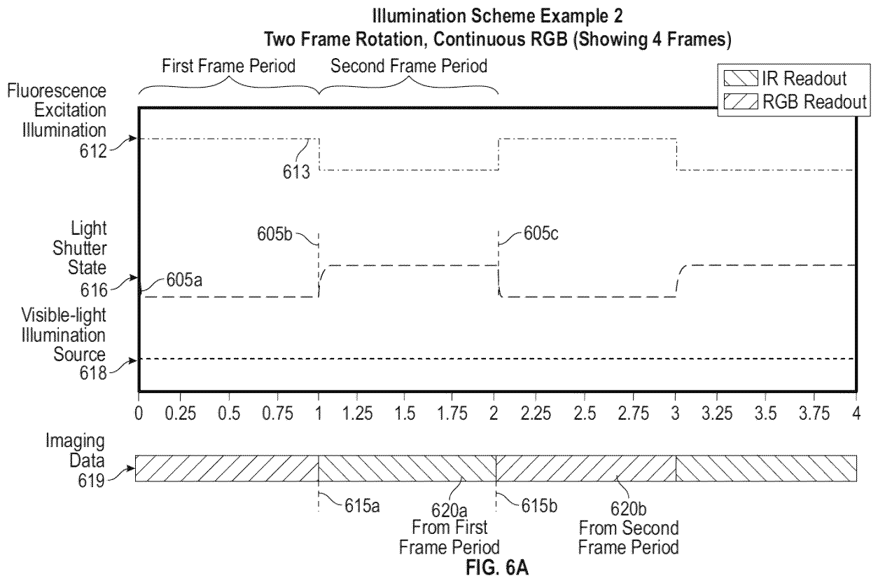

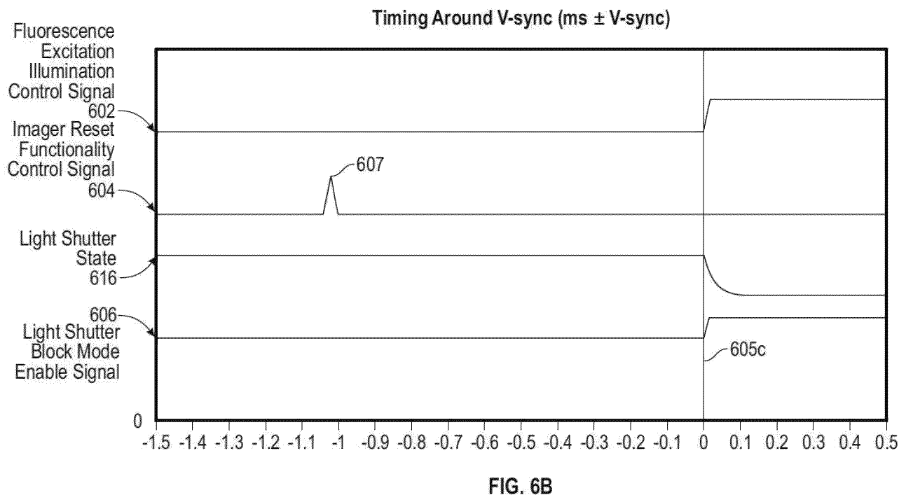

FIGS. 6A and 6B illustrate timing diagrams for operating an imaging system for imaging tissue from a subject, in accordance with some examples. In FIG. 6A, the illumination scheme involves a series of two-image rotations. Each rotation produces two images: a fluorescence image corresponding to a first frame period and a visible-light image corresponding to a second frame period. If the image sensor runs at 120 fps, this results in a 60 fps update of the output video, which is sufficient for continuous-appearing video without noticeable jerkiness.

With reference to FIG. 6A, the illumination level 612 of the fluorescence excitation illumination source is shown to comprise a sequence of fluorescence excitation illumination pulses, indicating that the fluorescence excitation illumination source is configured to provide fluorescence excitation illumination periodically for the duration of the pulse width shown in the illumination level 612. As one example of the illumination scheme, the fluorescence excitation illumination source can be pulsed at 60 Hz, turning on approximately at the vertical sync where the visible-light image readout begins, and off approximately at the next vertical sync. Further, the illumination level 618 of the visible-light illumination source is shown to be always on, indicating that the visible-light illumination source is configured to provide constant visible-light illumination during the imaging session. The light shutter state 616 can be open (shown as high) or closed (shown as low). As shown in FIG. 6A, the light shutter is configured to be in the open state during one frame period and to be in the closed state during the next frame period in an alternate manner. Specifically, the light shutter opens for the opposite frame as the fluorescence excitation illumination pulse; it opens during the entirety of the second frame period during which the visible-light image data is acquired and closes during the entirety of the first frame period during which the fluorescence image data is acquired.

At the beginning of the first frame period, at time 605a, the light shutter transitions from the open state to the closed state to prevent the global shutter imager from receiving any visible light. Specifically, the light shutter prevents the passage of light reflected from illumination of the tissue of the subject with the visible-light illumination pulse. As discussed above, while the light shutter is in the closed state, the light shutter still allows the passage of the light emitted due to illumination of the tissue of the subject with the fluorescence excitation illumination source to reach the global shutter imager. Thus, during the first frame period, the fluorescence excitation illumination source can illuminate the tissue of the subject with the fluorescence excitation illumination pulse 613 to accumulate charge at a plurality of pixels of the global shutter imager, while the light shutter is in the closed state.

At the end of the first frame period, at the readout time 615a, the imaging system begins reading out a first set of accumulated charge at the plurality of pixels of the global shutter imager to produce a first set of imaging data 620a. The first set of imaging data 620a contains only fluorescence imaging data because the light shutter prevented the global shutter imager from receiving any visible light during the first frame period. Accordingly, the imaging system generates a fluorescence image frame based on the first set of imaging data 620a. When readout begins, at readout time 605b, the charges have been transferred to holding capacitors and the pixels all reset, so the imager can begin accumulating the next image, and the imager may optionally be controlled to reset the charge part way through that period in order to adjust the exposure time (not depicted).

At the beginning of the second frame period, at time 605b, the light shutter transitions from the closed state to the open state to allow reflected light from illumination of the tissue of the subject with the visible-light illumination source to accumulate charge at the pixels of the global shutter imager. In the depicted example, the light shutter transitions from the closed state to the open state at time 605b and then transitions from the open state back to the closed state at time 605c, thus remaining open throughout the second frame period. While the light shutter is open, the reflected light from illumination of the tissue of the subject with the always-on visible-light illumination source causes accumulation of charge at the pixels of the global shutter imager. Further, during the illumination of the tissue of the subject with the visible-light illumination source, the fluorescence excitation illumination source is off.

At the end of the second frame period, at the readout time 615b, the imaging system begins reading a second set of accumulated charge at the plurality of pixels of the global shutter imager to produce a second set of imaging data 620b. The second set of imaging data 620b contains only visible-light imaging data because the light shutter was open to allow passage of visible light and the fluorescence excitation illumination source was off during the second frame period. Accordingly, the imaging system generates a visible-light image frame based on the second set of imaging data 620b.

Accordingly, the imaging system can generate a fluorescence image frame based on the first set of imaging data 620a and a visible-light image frame based on the second set of imaging data 620b. Another type of image frame can be a blended image frame based on both the fluorescence image frames and the visible-light image frame. In some examples, the fluorescence image frame can be overlaid on the visible-light image frame in the blended image frame. In some examples, the blended image frame can be derived from colorizing the visible-light image frame based on the fluorescence image frames (e.g., based on the ratio of the fluorescence image frame to one or more channels of the visible-light frame). One or more of the generated image frames can be added to a video stream and displayed on a display to a user (e.g., a medical practitioner such as a surgeon).

FIG. 6B illustrates a timing diagram with a zoomed-in view of the time around the end of the second frame period, in accordance with some examples. The x-axis of the timing diagram represents time in milliseconds relative to the vertical synchronization event (V-sync). FIG. 6B shows the alignment of the beginning of the fluorescence excitation illumination pulse with the V-sync and the timing of the light shutter. FIG. 6B illustrates control signals for operating various components of the imaging system, in accordance with some examples, including a control signal 602 for operating the fluorescence excitation illumination source, a control signal 604 for operating an optional reset functionality of the global shutter imager, and a control signal 606 for operating the light shutter.

With reference to FIG. 6B, the control signal 606 for operating the light shutter controls the timing for transitioning the light shutter between the open state and the closed state. In the depicted example, the control signal 606 dictates when to enable a “block mode (i.e., the closed state) of the light shutter. At any given time, the control signal 606 can be either high (indicating that the “block mode” is enabled) or low (indicating that the “block mode” is disabled). Further with reference to FIG. 6B, the control signal 604 controls the timing for activating an optional reset functionality of the global shutter imager. All rows of pixels in the image sensor can be reset simultaneously or within a short time period upon triggering the control signal 604 (e.g., in addition to being automatically reset with each readout).

As shown in FIG. 6B, at the time 605c, the control signal 606 for operating the light shutter changes from low (i.e., block mode disabled) to high (i.e., block mode enabled). In response to the control signal 606, the light shutter starts to transition from the open state to the closed state starting at time 605a, as shown by the rapid downward curvature of the light shutter state 616 starting from the time 605a. The gradual downward curvature of the light shutter state 616 indicates that the light shutter does not instantly transition from the open state to the closed state. Rather, the transitioning is gradual over a very short time and thus the amount of light passage decreases over that time. It should be appreciated by one of ordinary skill in the art that the gradual nature of the transitioning is due to the nature of the liquid crystal material.

Notably in FIG. 6B, the control signal 604 for operating the reset functionality triggers at time 607, resetting of all rows of pixels in the image sensor of the global shutter imager simultaneously or within a short time period. The time 607 for triggering the reset functionality can occur any time during the second frame period to control the exposure time of the global shutter sensor and thus the brightness of the visible-light image. In all of the examples described herein, a different control signal can be used during the fluorescence imaging data acquisition period to control the brightness of the fluorescence image frame.

The example in FIGS. 6A-B provides several technical advantages. The two-frame rotation allows for continuous 60 Hz output with a 120 Hz image sensor. All of the fluorescence excitation illumination is used for imaging fluorescence, so there is no wasted fluorescence excitation illumination. Further, by pulsing the fluorescence excitation illumination source rather than running it continuously, there is no need to correct for fluorescence in the visible-light image frame. The continuous visible-light illumination is guaranteed to appear continuous to the eye and allows for the global reset pulse 604 to be anywhere during the frame period (rather than only during a primary visible-light illumination pulse), allowing for greater dynamic range of the visible-light image. Further, the fluorescence imaging data is not contaminated by any visible light and thus there is no need to acquire ambient visible-light imaging data.

### Example Three of the Illumination Scheme

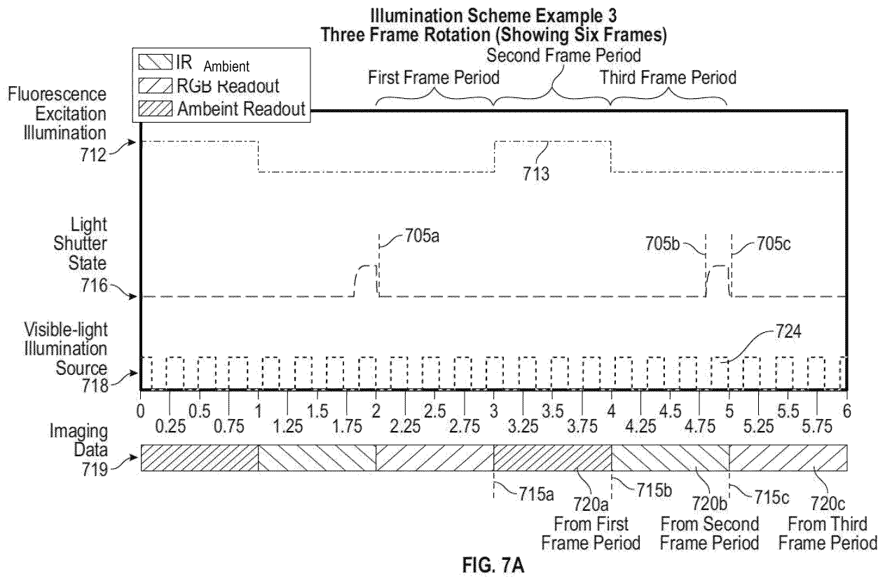

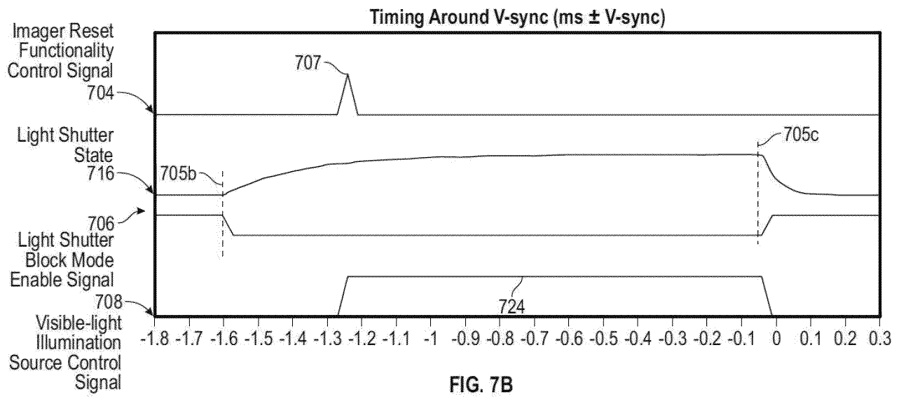

FIGS. 7A and 7B illustrate timing diagrams for operating an imaging system for imaging tissue from a subject, in accordance with some examples. In FIG. 7A, the illumination scheme involves a series of three-image rotations. Each rotation produces three images: an ambient frame in a first frame period, a fluorescence image frame in a second frame period, and a visible-light image corresponding to a third frame period.

With reference to FIG. 7A, the illumination level 712 of the fluorescence excitation illumination source is shown to comprise a sequence of fluorescence excitation illumination pulses, indicating that the fluorescence excitation illumination source is configured to provide fluorescence excitation illumination periodically for the duration of the pulse width shown in the illumination level 712. As one example of the illumination scheme, the fluorescence excitation illumination source can be pulsed at 40 Hz (or faster if the sensor runs faster than 120 fps), turning on approximately at the vertical sync one frame period after the primary visible-light illumination pulse ends, and turning off approximately at the next vertical sync. If the sensor runs at 180 fps, then the fluorescence excitation would be pulsed at 60 Hz and the video output would be updated at 60 fps, which is sufficient for continuous-appearing video without noticeable jerkiness.

Further, the illumination level 718 of the visible-light illumination source is shown to comprise a sequence of visible-light illumination pulses, indicating that the visible-light illumination source is configured to provide visible-light illumination periodically for the duration of the pulse width shown in the illumination level 718. In the depicted example, the visible-light illumination source is pulsed, with one primary pulse ending approximately at the vertical sync of the image sensor, on every third frame, and one or more additional compensation pulses equally spaced between the primary pulses. In this example, there are 10 compensation pulses between neighboring primary visible-light illumination pulses, for a total of 11 pulses (440 Hz, if the sensor is running at 120 fps), but there can be as few as one compensation pulse between neighboring primary visible-light illumination pulses. The maximum number can be bounded by the off period needing to be longer than the ramp-open time of the light shutter, and the on period needing to be long enough to acquire a reasonably bright image. The minimum number can be bounded to one (which is equivalent to 80 Hz, if the sensor is running at 120 fps) to ensure that the illumination looks continuous to the eye.

The light shutter state 716 can be open (shown as high) or closed (shown as low). As shown in FIG. 7A, the light shutter is generally in the closed state but is transitioned into the open state periodically. Specifically, the light shutter opens only around the primary visible-light illumination pulse.

At the beginning of the first frame period, at time 705a, the light shutter transitions from the open state to the closed state to prevent the global shutter imager from receiving any visible light. Specifically, the light shutter prevents the passage of light reflected from illumination of the tissue of the subject with the visible-light illumination pulse and also prevents passage of any ambient visible light. Further, the fluorescence excitation illumination source is off during the first frame period. Thus, during the first frame period, the global shutter imager does not receive any visible light; nor does it receive any light emitted due to illumination with the fluorescence excitation illumination source because the illumination source is off.

However, during the first frame period, the light shutter still allows the passage of the light emitted due to illumination of the tissue of the subject with ambient light in the fluorescence emission band. Thus, during the first frame period, the tissue of the subject is illuminated with ambient light in the fluorescence band, causing accumulation of charge at a plurality of pixels of the global shutter imager.

At the end of the first frame period (i.e., at readout time 715a), the imaging system begins reading a first set of accumulated charge at the plurality of pixels of the global shutter imager to produce a first set of imaging data 720a. The first set of imaging data 720a contains only imaging data from ambient light in the fluorescence emission band, because the light shutter prevented the global shutter imager from receiving any visible light during the first frame period and the fluorescence excitation illumination source was off. Accordingly, the imaging system generates an ambient image frame based on the first set of imaging data 720a. When readout begins at the readout time 715a, the charges have been transferred to holding capacitors and the pixels all reset, so the imager can begin accumulating the next image, and the imager may optionally be controlled to reset the charge part way through that period in order to adjust the exposure time (not depicted).

During the second frame period, the light shutter remains in the closed state as shown by the light shutter state 716 to continue to prevent the global shutter imager from receiving any visible light. However, the florescence excitation illumination source is on during the second frame period, as shown by the fluorescence excitation illumination pulse 713. The light shutter still allows the passage of the light emitted due to illumination of the tissue of the subject with both ambient illumination and the fluorescence excitation illumination pulse 713, causing accumulation of charge at a plurality of pixels of the global shutter imager.

At the end of the second frame period (i.e., at readout time 715b), the imaging system begins reading out a second set of accumulated charge at the plurality of pixels of the global shutter imager to produce a second set of imaging data 720b. The second set of imaging data 720b contains fluorescence imaging data because the light shutter prevented the global shutter imager from receiving any visible light during the second frame period, but the tissue of the subject is illuminated with both the fluorescence excitation illumination source and ambient illumination. Accordingly, the imaging system generates a fluorescence image frame based on the second set of imaging data 720b. When readout begins at the readout time 715b, the charges have been transferred to holding capacitors and the pixels all reset, so the imager can begin accumulating the next image, and the imager may optionally be controlled to reset the charge part way through that period in order to adjust the exposure time (not depicted).

During the third frame period, at time 705b, the light shutter transitions from the closed state to the open state to allow reflected light from illumination of the tissue of the subject with the visible-light illumination source to accumulate charge at the pixels of the global shutter imager. In the depicted example, the light shutter transitions from the closed state to the open state at time 705b and then transitions from the open state back to the closed state at time 705c. While the light shutter is open, the reflected light from illumination of the tissue of the subject with the visible-light illumination pulse 724 accumulates charge at the pixels of the global shutter imager. Further, during the illumination of the tissue of the subject with the visible-light illumination pulse 724, the fluorescence excitation illumination source is off.

At the end of the third frame period (i.e., at the readout time 715c), the imaging system begins reading out a set of accumulated charge at the plurality of pixels of the global shutter imager to produce a third set of imaging data 720c. The third set of imaging data 720c contains only visible-light imaging data and ambient light imaging data (which is negligible in the combined data due to the dominating visible-light imaging data) because the light shutter was open and the fluorescence illumination excitation source was off during the third frame period. Accordingly, the imaging system generates a visible-light image frame based on the third set of imaging data 720c.

Accordingly, the imaging system can generate an ambient image frame based on the first set of imaging data 720a, a fluorescence image frame based on the second set of imaging data 720b, and a visible-light image frame based on the third set of imaging data 720c. The system can subtract the ambient image frame from the fluorescence image frame to obtain a final fluorescence image frame for display. Another type of image frame can be a blended image frame based on both the final fluorescence image frames and the visible-light image frame. In some examples, the final fluorescence image frame can be overlaid on the visible-light image frame in the blended image frame. In some examples, the blended image frame can be derived from colorizing the visible-light image frame based on the final fluorescence image frames (e.g., based on the ratio of the fluorescence image frame to one or more channels of the visible-light frame). One or more of the generated image frames can be added to a video stream and displayed on a display to a user (e.g., a medical practitioner such as a surgeon).

FIG. 7B illustrates a timing diagram with a zoomed-in view of the time period around the end of the third frame period, in accordance with some examples. The x-axis of the timing diagram represents time in milliseconds relative to a vertical synchronization event (V-sync). FIG. 7B illustrates control signals for operating various components of the imaging system, in accordance with some examples, including a control signal 704 for operating an optional reset functionality of the global shutter imager, a control signal 706 for operating the light shutter, and a control signal 708 for operating the visible-light illumination source.

With reference to FIG. 7B, the control signal 706 for operating the light shutter controls the timing for transitioning the light shutter between the closed state and the open state. In the depicted example, the control signal 706 dictates when to enable a “block mode (i.e., the closed state) of the light shutter. At any given time, the control signal 706 can be either high (indicating that the “block mode” is enabled) or low (indicating that the “block mode” is disabled). Further with reference to FIG. 7B, the control signal 704 controls the timing for activating an optional reset functionality of the global shutter imager. All rows of pixels in the image sensor can be reset simultaneously or within a short time period upon triggering the control signal 704 (e.g., in addition to being automatically reset with each readout).

As shown in FIG. 7B, at the time 705b, the control signal 706 for operating the light shutter changes from high (i.e., block mode enabled) to low (i.e., block mode disabled). In response to the control signal 706, the light shutter starts to transition from the closed state to the open state starting at time 705b, as shown by the gradual upward curvature of the light shutter state 716 starting from the time 705b. The gradual upward curvature of the light shutter state 716 indicates that the light shutter does not instantly transition from the closed state to the open state. Rather, the transitioning is gradual over time and thus the amount of light passage gradually increases over time during the transitioning. It should be appreciated by one of ordinary skill in the art that the gradual nature of the transitioning is due to the nature of the liquid crystal material.

Then at the time 705c, the control signal 706 for operating the light shutter changes from low (i.e., block mode disabled) to high (i.e., block mode enabled). In response to the control signal 706, the light shutter starts to transition from the open state to the closed state starting at the time 705c. The rapid downward curvature of the light shutter state 716 indicates that light shutter does not instantly transition from the open state to the closed state. Rather, the transitioning is gradual over a very short time and thus the amount of light passage gradually over that time due to the nature of the liquid crystal material.

Notably in FIG. 7B, the visible-light illumination source is activated to provide a visible-light illumination pulse 724 after the time 705b. As shown in FIG. 7B, the transitioning of the light shutter from the closed state to the open state is complete or substantially complete when the visible-light illumination pulse 724 starts, thus allowing the passage of the light reflected from illumination of the tissue of the subject with the visible-light illumination pulse 724 to reach the global shutter imager. Further, the control signal 704 for operating the reset functionality triggers at time 707, resetting of all rows of pixels in the image sensor of the global shutter imager simultaneously or within a short time period. Using the control signal 704, the exposure period for the visible-light frame is adjusted to maintain image brightness but is restricted to no longer than the duration of the primary visible-light pulse. Specifically, the control signal 704 can be anywhere between the time 707 and V-sync.

Further in FIG. 7B, the visible-light illumination source is deactivated to end the visible-light illumination pulse 724 at the same as the time 705c when the light shutter starts to transition to the closed state. This way, the transitioning of the light shutter to the closed state would not block the passage of the light reflected from illumination of the tissue of the subject with the visible-light illumination pulse 724. Further, the transitioning of the light shutter from the open state to the closed state is complete before the next visible-light illumination pulse starts. As discussed herein, the next visible-light illumination pulse is a compensating pulse and is blocked by the light shutter from reaching the global shutter imager.

The examples in FIGS. 7A-B provide several technical advantages. Three-frame rotation allows for compensation of ambient light signals (e.g., from surgical lights). All of the fluorescence excitation illumination is used for imaging fluorescence, so there is no wasted fluorescence excitation illumination. Further, by pulsing the fluorescence excitation illumination source rather than running it continuously, there is no need to correct for fluorescence in the visible-light image frame. Further, the fluorescence imaging data is not contaminated by any visible light and thus there is no need to acquire ambient visible-light imaging data. While the longer rotation (3 frames vs. 2 frames) reduces the output image update rate to 40 fps instead of the standard 60 fps, this drawback can be reduced by running the sensor at 180 fps instead of 120 fps, and scaling all of the illumination timing accordingly.

### Example Four of the Illumination Scheme

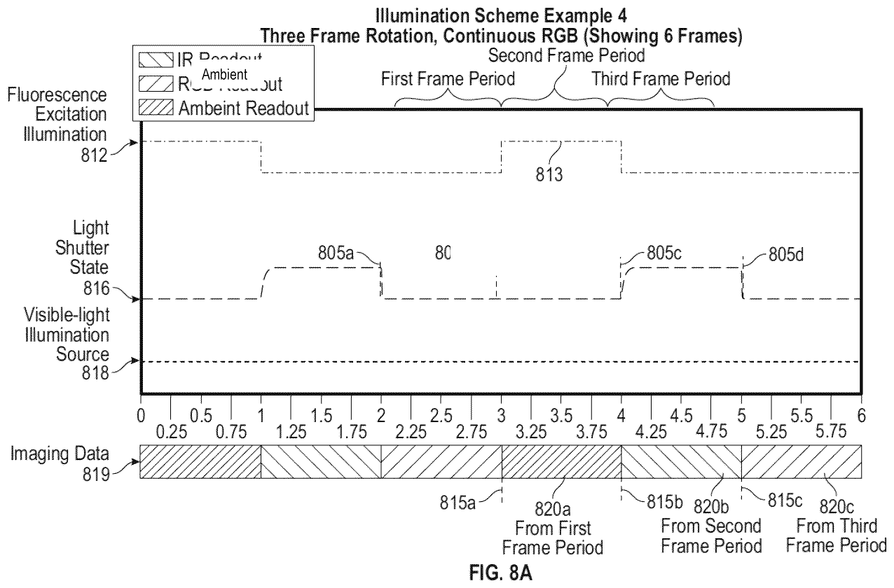

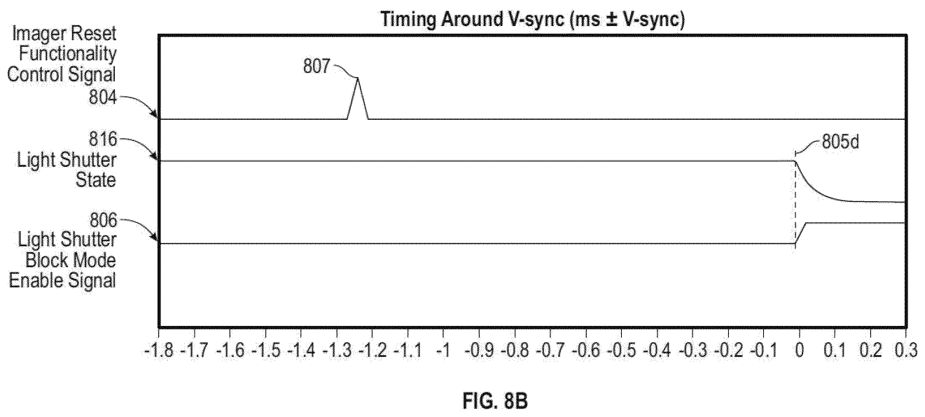

FIGS. 8A and 8B illustrate timing diagrams for operating an imaging system for imaging tissue from a subject, in accordance with some examples. In FIG. 8A, the illumination scheme involves a series of three-image rotations. Each rotation produces three images: an ambient frame in a first frame period, a fluorescence image frame in a second frame period, and a visible-light image corresponding to a third frame period.

With reference to FIG. 8A, the illumination level 812 of the fluorescence excitation illumination source is shown to comprise a sequence of fluorescence excitation illumination pulses, indicating that the fluorescence excitation illumination source is configured to provide fluorescence excitation illumination periodically for the duration of the pulse width shown in the illumination level 812. As one example of the illumination scheme, the fluorescence excitation illumination source can be pulsed at 40 Hz (or faster if the sensor runs faster than 120 fps), turning on approximately at the vertical sync where the visible-light image readout begins, and turning off approximately at the next vertical sync. If the sensor runs at 180 fps, then the fluorescence excitation would be pulsed at 60 Hz and the video output would be updated at 60 fps, which is sufficient for continuous-appearing video without noticeable jerkiness. Further, the illumination level 818 of the visible-light illumination source is also shown to be always on, indicating that the visible-light illumination source is configured to provide constant visible-light illumination for the imaging session. The light shutter state 816 can be open (shown as high) or closed (shown as low). As shown in FIG. 8A, the light shutter is configured to be open in the first frame period and closed in the subsequent second frame period and third frame period.

At the beginning of the first frame period, at time 805a, the light shutter transitions from the open state to the closed state to prevent the global shutter imager from receiving any visible light. Specifically, the light shutter prevents the passage of light reflected from illumination of the tissue of the subject with the visible-light illumination pulse and also prevents passage of any ambient visible light. Further, the fluorescence excitation illumination source is off during the first frame period. Thus, during the first frame period, the global shutter imager does not receive any visible light; nor does it receive any light emitted due to illumination with the fluorescence excitation illumination source because the illumination source is off.

However, during the first frame period, the light shutter still allows the passage of the light emitted due to illumination of the tissue of the subject with ambient illumination in the fluorescence band. Thus, during the first frame period, the tissue of the subject is illuminated with ambient illumination in the fluorescence band, causing accumulation of charge at a plurality of pixels of the global shutter imager.

At the end of the first frame period (i.e., at readout time 815a), the imaging system begins reading out a first set of accumulated charge at the plurality of pixels of the global shutter imager to produce a first set of imaging data 820a. The first set of imaging data 820a contains only ambient light in the fluorescence band imaging data because the light shutter prevented the global shutter imager from receiving any visible light during the first frame period and the fluorescence excitation illumination source was off. Accordingly, the imaging system generates an ambient image frame based on the first set of imaging data 820a. When readout begins at the readout time 815a, the charges have been transferred to holding capacitors and the pixels all reset, so the imager can begin accumulating the next image, and the imager may optionally be controlled to reset the charge part way through that period in order to adjust the exposure time (not depicted).

During the second frame period, the light shutter remains in the closed state as shown by the light shutter state 816 to continue to prevent the global shutter imager from receiving any visible light. However, the florescence excitation illumination source is on during the second frame period, as shown by the fluorescence excitation illumination pulse 813. The light shutter still allows the passage of the light emitted due to illumination of the tissue of the subject with both ambient illumination in the fluorescence emission band and the fluorescence excitation illumination pulse 813, causing accumulation of charge at a plurality of pixels of the global shutter imager.

At the end of the second frame period (i.e., at readout time 815b), the imaging system begins reading out a second set of accumulated charge at the plurality of pixels of the global shutter imager to produce a second set of imaging data 820b. The second set of imaging data 820b contains fluorescence imaging data because the light shutter prevented the global shutter imager from receiving any visible light during the second frame period, but the tissue of the subject is illuminated with both the fluorescence excitation illumination source and ambient illumination in the fluorescence emission band. Accordingly, the imaging system generates a fluorescence image frame based on the second set of imaging data 820b. When readout begins at the readout time 815b, the charges have been transferred to holding capacitors and the pixels all reset, so the imager can begin accumulating the next image, and the imager may optionally be controlled to reset the charge part way through that period in order to adjust the exposure time (not depicted).

At the beginning of the third frame period, at time 815b, the light shutter transitions from the closed state to the open state to allow reflected light from illumination of the tissue of the subject with the visible-light illumination source to accumulate charge at the pixels of the global shutter imager. In the depicted example, the light shutter transitions from the closed state to the open state at time 805c and then transitions from the open state back to the closed state at time 805d. While the light shutter is open, the reflected light from illumination of the tissue of the subject with the always-on visible-light illumination source reaches the global shutter imager to cause accumulation of charge at the pixels of the global shutter imager. Further, during the illumination of the tissue of the subject with the visible-light illumination source, the fluorescence excitation illumination source is off in the third frame period.

At the end of the third frame period (i.e., at the readout time 815c), the imaging system begins reading out a set of accumulated charge at the plurality of pixels of the global shutter imager to produce a third set of imaging data 820c. The third set of imaging data 820c contains only visible-light imaging data because the light shutter was open and the fluorescence excitation illumination source was off during the third frame period. Accordingly, the imaging system generates a visible-light image frame based on the third set of imaging data 820c.

Accordingly, the imaging system can generate an ambient image frame based on the first set of imaging data 820a, a fluorescence image frame based on the second set of imaging data 820b, and a visible-light image frame based on the third set of imaging data 820c. The system can subtract the ambient image frame from the fluorescence image frame to obtain a final fluorescence image frame for display. Another type of image frame can be a blended image frame based on both the final fluorescence image frames and the visible-light image frame. In some examples, the final fluorescence image frame can be overlaid on the visible-light image frame in the blended image frame. In some examples, the blended image frame can be derived from colorizing the visible-light image frame based on the final fluorescence image frames (e.g., based on the ratio of the fluorescence image frame to one or more channels of the visible-light frame). One or more of the generated image frames can be added to a video stream and displayed on a display to a user (e.g., a medical practitioner such as a surgeon).

FIG. 8B illustrates a timing diagram with a zoomed-in view of the time around the end of the third frame period, in accordance with some examples. The x-axis of the timing diagram represents time in milliseconds relative to the vertical synchronization event (V-sync). FIG. 8B illustrates control signals for operating various components of the imaging system, in accordance with some examples, including a control signal 804 for operating an optional reset functionality of the global shutter imager and a control signal 806 for operating the light shutter.

With reference to FIG. 8B, the control signal 806 for operating the light shutter controls the timing for transitioning the light shutter between the open state and the closed state. In the depicted example, the control signal 806 dictates when to enable a “block mode (i.e., the closed state) of the light shutter. At any given time, the control signal 806 can be either high (indicating that the “block mode” is enabled) or low (indicating that the “block mode” is disabled). Further with reference to FIG. 8B, the control signal 804 controls the timing for activating an optional reset functionality of the global shutter imager. All rows of pixels in the image sensor can be reset simultaneously or within a short time period upon triggering the control signal 804 (e.g., in addition to being automatically reset with each reset).

As shown in FIG. 8B, at the time 805d, the control signal 806 for operating the light shutter changes from low (i.e., block mode disabled) to high (i.e., block mode enabled). In response to the control signal 806, the light shutter starts to transition from the open state to the closed state starting at time 805d, as shown by the rapid downward curvature of the light shutter state 816 starting from the time 805d. The downward curvature of the light shutter state 816 indicates that the light shutter does not instantly transition from the open state to the closed state. Rather, the transitioning is gradual over a very short time and thus the amount of light passage gradually decreases over that time during the transitioning. It should be appreciated by one of ordinary skill in the art that the gradual nature of the transitioning is due to the nature of the liquid crystal material.

Notably in FIG. 8B, the control signal 804 for operating the reset functionality triggers at time 807, resetting of all rows of pixels in the image sensor of the global shutter imager simultaneously or within a short time period. The global reset pulse, which controls the exposure time of the global shutter sensor, could be anywhere during the frame, with that adjustment being used to control the visible-light image brightness. Global reset pulses could be used during the ambient and fluorescence acquisition periods to adjust the brightness of the fluorescence frame. If different exposure periods are used, then the ambient subtraction needs to be scaled according to the ratio of exposure periods.

The examples in FIGS. 8A-B provide several technical advantages. Three-frame rotation allows for compensation of ambient signals in the fluorescence band (e.g., from surgical lights or other NIR sources). All of the fluorescence excitation illumination is used for imaging fluorescence, so there is no wasted fluorescence excitation illumination. Further, by pulsing the fluorescence excitation illumination source rather than running it continuously, there is no need to correct for fluorescence in the visible-light image frame. Continuous visible-light illumination is guaranteed to look continuous to the eye and allows for the global reset pulse to be anywhere during the frame, allowing for greater dynamic range of the visible-light image. Further, the fluorescence imaging data is not contaminated by any visible light and thus there is no need to acquire ambient visible-light imaging data. While the longer rotation (3 frames vs. 2 frame) reduces the output image update rate to 40 fps instead of the standard 60 fps, this drawback can be reduced by running the sensor at 180 fps instead of 120 fps, and scaling all of the illumination timing accordingly.

It should be appreciated by one of ordinary skill in the art that the examples in FIGS. 5A-8B are merely exemplary. For example, the order in which the fluorescence image data, the visible-light image data, and/or the ambident fluorescence image data are acquired can be changed. Further, in some examples, the imaging system can be configured to capture only fluorescence images without capturing visible-light images, or capture both types of images but display only fluorescence images. In any of the examples described herein, the techniques described herein can be used for NIR reflectance imaging without fluorescence excitation imaging.

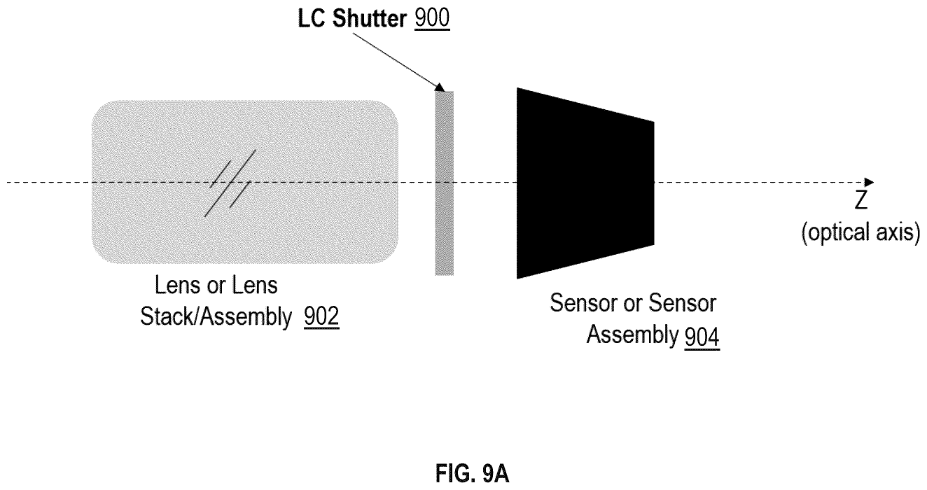

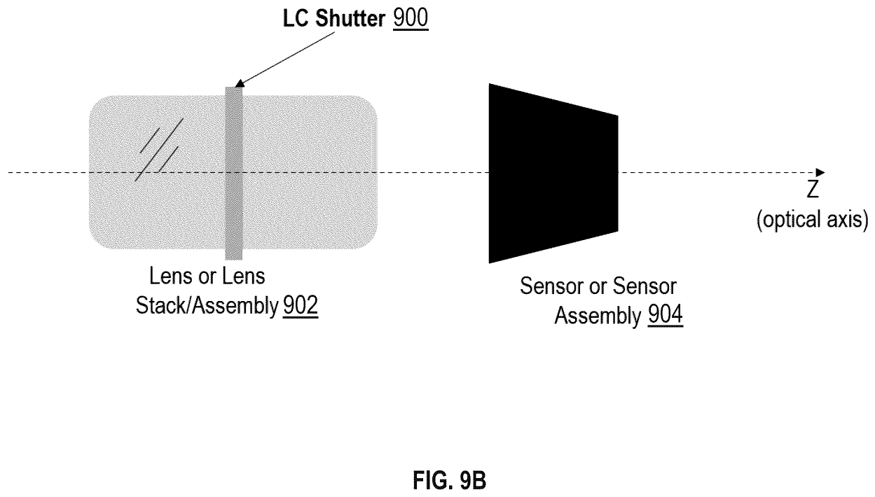

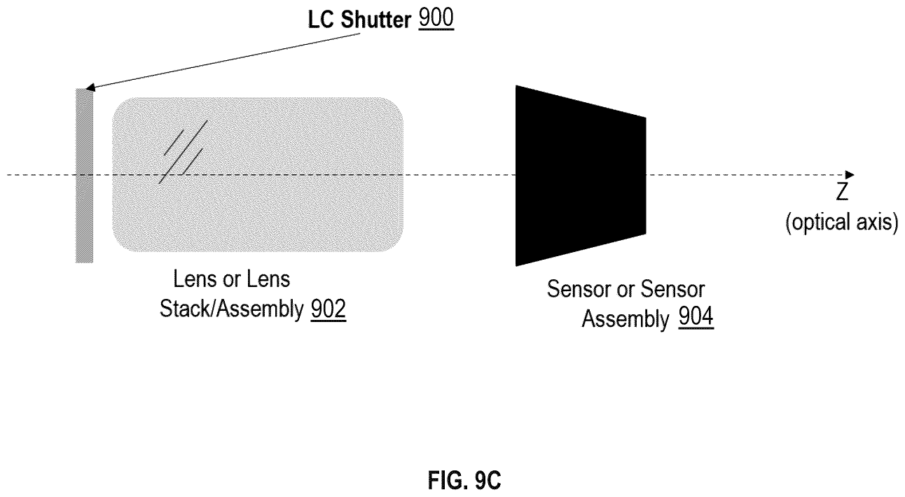

FIGS. 9A-9C illustrate exemplary locations of the liquid crystal light shutter, in accordance with some examples. In FIG. 9A, the LC shutter 900 is located between the lens or lens stack assembly 902 and the sensor or sensor assembly 904. In FIG. 9B, the LC shutter 900 is located within the lens stack or lens stack assembly 902. In FIG. 9C, the LC shutter 900 is located before the lens stack or lens stack assembly 902. In some examples, the LC shutter is preferably located as close to the sensor or sensor assembly as possible. For example, the configuration of FIG. 9A may be preferable to the configuration of FIG. 9C.

### EMBODIMENTS

The present disclosure includes at least the following embodiments.
1. A method of imaging tissue of a subject using a fluorescence imaging system comprising a global shutter imager, a liquid crystal light shutter configurable to be in an open state and a closed state, and a fluorescence excitation illumination source, the method comprising:
   transitioning the liquid crystal light shutter to the closed state to prevent the global shutter imager from receiving visible light;
   illuminating the tissue of the subject with the fluorescence excitation illumination source to accumulate charge at a plurality of pixels of the global shutter imager, while the liquid crystal light shutter is in the closed state;
   reading a set of accumulated charge at the plurality of pixels of the global shutter imager to produce a set of imaging data; and
   generating a fluorescence image frame based on the set of imaging data.
2. The method of embodiment 1, further comprising: displaying the generated fluorescence image by adding the fluorescence image frame to a video stream.
3. The method of embodiment 1,
   wherein the fluorescence imaging system further comprises a visible-light illumination source,
   wherein the set of accumulated charge is a first set of accumulated charge, and
   wherein the set of imaging data is a first set of imaging data,
   the method further comprising:
      transitioning the liquid crystal light shutter to the open state to allow reflected light from illumination of the tissue of the subject with the visible-light illumination source to accumulate charge at the pixels of the global shutter imager;
      reading a second set of accumulated charge at the plurality of pixels of the global shutter imager to produce a second set of imaging data; and
      generating a visible-light image frame based on the second set of imaging data.
4. The method of embodiment 3, wherein the fluorescence excitation illumination source is off during the illumination of the tissue of the subject with the visible-light illumination source.
5. The method of embodiment 3 or embodiment 4, further comprising: generating a blended image frame based on the fluorescence image frame and the visible-light image frame.
6. The method of embodiment 5, wherein the fluorescence image frame is overlaid on the visible-light image frame in the blended image frame.
7. The method of embodiment 5, wherein the blended image frame is derived from colorizing the visible-light image frame based on the fluorescence image frame.
8. The method of embodiment 7, wherein the blended image frame is derived from colorizing the visible-light image frame based on the ratio of the fluorescence image frame to one or more channels of the visible-light frame.
9. The method of any of embodiments 5-8, further comprising: displaying the blended image frame by adding the blended image frame to a video stream.
10. The method of any of embodiments 3-9, wherein the visible-light illumination source is pulsed.
11. The method of embodiment 10, wherein the pulsed visible-light illumination source is configured to include:
   a plurality of primary visible-light illumination pulses for illuminating the tissue of the subject while the liquid crystal light shutter is in the open state; and
   one or more compensating visible-light illumination pulses between each two neighboring primary visible-light illumination pulses.
12. The method of embodiment 11, wherein the liquid crystal light shutter is in the closed state during the one or more compensating visible-light illumination pulses.
13. The method of any of embodiments 10-12, further comprising:
   while the fluorescence excitation illumination source is off and the liquid crystal light shutter is in the closed state, illuminating the tissue of the subject with ambient illumination to accumulate charge at the plurality of pixels of the global shutter imager;
   reading a third set of accumulated charge at the plurality of pixels of the global shutter imager to produce a third set of imaging data; and
   generating an ambient image frame based on the third set of imaging data.
14. The method of embodiment 13, wherein the ambient illumination comprises ambient illumination in the fluorescence emission band.
15. The method of embodiment 13 or 14, further comprising: subtracting the ambient image frame from the fluorescence image frame.
16. The method of any of embodiments 3-9, wherein the visible-light illumination source is continuous.
17. The method of embodiment 16, further comprising:
   while the fluorescence excitation illumination source is off and the liquid crystal light shutter is in the closed state, illuminating the tissue of the subject with ambient illumination to accumulate charge at the plurality of pixels of the global shutter imager;
   reading a third set of accumulated charge at the plurality of pixels of the global shutter imager to produce a third set of imaging data; and
   generating an ambient image frame based on the third set of imaging data.
18. The method of embodiment 17, wherein the ambient illumination comprises ambient illumination in the fluorescence emission band.
19. The method of embodiment 17 or 18, further comprising: subtracting the ambient image frame from the fluorescence image frame.
20. A system of imaging tissue of a subject, the system comprising:
   a fluorescence excitation illumination source,
   a visible-light illumination source,
   a liquid crystal light shutter configurable to be in an open state and a closed state, and an imager being configured for:
      transitioning the liquid crystal light shutter to the closed state to prevent the global shutter imager from receiving visible light;
      illuminating the tissue of the subject with the fluorescence excitation illumination source to accumulate charge at a plurality of pixels of the global shutter imager, while the liquid crystal light shutter is in the closed state;
      reading a set of accumulated charge at the plurality of pixels of the global shutter imager to produce a set of imaging data; and
      generating a fluorescence image frame based on the set of imaging data.
21. The system of embodiment 20, wherein the imager is further configured for: displaying the generated fluorescence image by adding the fluorescence image frame to a video stream.
22. The system of embodiment 20,
   wherein the fluorescence imaging system further comprises a visible-light illumination source,
   wherein the set of accumulated charge is a first set of accumulated charge, and
   wherein the set of imaging data is a first set of imaging data, and
   wherein the imager is further configured for:
      transitioning the liquid crystal light shutter to the open state to allow reflected light from illumination of the tissue of the subject with the visible-light illumination source to accumulate charge at the pixels of the global shutter imager;
      reading a second set of accumulated charge at the plurality of pixels of the global shutter imager to produce a second set of imaging data; and
      generating a visible-light image frame based on the second set of imaging data.
23. The system of embodiment 22, wherein the fluorescence excitation illumination source is off during the illumination of the tissue of the subject with the visible-light illumination source.
24. The system of embodiment 22, wherein the imager is further configured for: generating a blended image frame based on the fluorescence image frame and the visible-light image frame.
25. The system of embodiment 24, wherein the fluorescence image frame is overlaid on the visible-light image frame in the blended image frame.
26. The system of embodiment 24, wherein the blended image frame is derived from colorizing the visible-light image frame based on the fluorescence image frame.
27. The system of embodiment 26, wherein the blended image frame is derived from colorizing the visible-light image frame based on the ratio of the fluorescence image frame to one or more channels of the visible-light frame.
28. The system of any of embodiments 24-27, wherein the imager is further configured for: displaying the blended image frame by adding the blended image frame to a video stream.
29. The system of any of embodiments 22-28, wherein the visible-light illumination source is pulsed.
30. The system of embodiment 29, wherein the pulsed visible-light illumination source is configured to include:
   a plurality of primary visible-light illumination pulses for illuminating the tissue of the subject while the liquid crystal light shutter is in the open state; and
   one or more compensating visible-light illumination pulses between each two neighboring primary visible-light illumination pulses.
31. The system of embodiment 30, wherein the liquid crystal light shutter is in the closed state during the one or more compensating visible-light illumination pulses.
32. The system of any of embodiments 29-31, wherein the imager is further configured for:
   while the fluorescence excitation illumination source is off and the liquid crystal light shutter is in the closed state, illuminating the tissue of the subject with ambient illumination to accumulate charge at the plurality of pixels of the global shutter imager;
   reading a third set of accumulated charge at the plurality of pixels of the global shutter imager to produce a third set of imaging data; and
   generating an ambient image frame based on the third set of imaging data.
33. The system of embodiment 32, wherein the ambient illumination comprises light in the fluorescence emission band.
34. The system of embodiment 32 or 33, wherein the imager is further configured for: subtracting the ambient image frame from the fluorescence image frame.
35. The system of any of embodiments 22-28, wherein the visible-light illumination source is continuous.
36. The system of embodiment 35, wherein the imager is further configured for:
   while the fluorescence excitation illumination source is off and the liquid crystal light shutter is in the closed state, illuminating the tissue of the subject with ambient illumination to accumulate charge at the plurality of pixels of the global shutter imager;
   reading a third set of accumulated charge at the plurality of pixels of the global shutter imager to produce a third set of imaging data; and
   generating an ambient image frame based on the third set of imaging data.
37. The system of embodiment 36, wherein the ambient illumination comprises ambient light in the fluorescence emission band.
38. The system of embodiment 36 or 37, wherein the imager is further configured for: subtracting the ambient image frame from the fluorescence image frame.

The foregoing description, for the purpose of explanation, has been described with reference to specific examples. However, the illustrative discussions above are not intended to be exhaustive or to limit the invention to the precise forms disclosed. For the purpose of clarity and a concise description, features are described herein as part of the same or separate examples; however, it will be appreciated that the scope of the disclosure includes examples having combinations of all or some of the features described. Many modifications and variations are possible in view of the above teachings. The examples were chosen and described in order to best explain the principles of the techniques and their practical applications. Others skilled in the art are thereby enabled to best utilize the techniques and various examples with various modifications as are suited to the particular use contemplated.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims. Finally, the entire disclosure of the patents and publications referred to in this application are hereby incorporated herein by reference.

What is claimed is:

1. A method of imaging tissue of a subject using a fluorescence imaging system comprising a global shutter imager, a liquid crystal light shutter configurable to be in an open state and a closed state, and a fluorescence excitation illumination source, the method comprising:
transitioning the liquid crystal light shutter to the closed state to prevent the global shutter imager from receiving visible light;
illuminating the tissue of the subject with the fluorescence excitation illumination source to accumulate charge at a plurality of pixels of the global shutter imager, while the liquid crystal light shutter is in the closed state;
reading a set of accumulated charge at the plurality of pixels of the global shutter imager to produce a set of imaging data; and
generating a fluorescence image frame based on the set of imaging data.

2. The method of claim 1, further comprising: displaying the generated fluorescence image by adding the fluorescence image frame to a video stream.

3. The method of claim 1,
wherein the fluorescence imaging system further comprises a visible-light illumination source,
wherein the set of accumulated charge is a first set of accumulated charge, and
wherein the set of imaging data is a first set of imaging data,
the method further comprising:
transitioning the liquid crystal light shutter to the open state to allow reflected light from illumination of the tissue of the subject with the visible-light illumination source to accumulate charge at the pixels of the global shutter imager;
reading a second set of accumulated charge at the plurality of pixels of the global shutter imager to produce a second set of imaging data; and
generating a visible-light image frame based on the second set of imaging data.

4. The method of claim 3, wherein the fluorescence excitation illumination source is off during the illumination of the tissue of the subject with the visible-light illumination source.

5. The method of claim 3, further comprising: generating a blended image frame based on the fluorescence image frame and the visible-light image frame.

6. The method of claim 5, wherein the fluorescence image frame is overlaid on the visible-light image frame in the blended image frame.

7. The method of claim 5, wherein the blended image frame is derived from colorizing the visible-light image frame based on the fluorescence image frame.

8. The method of claim 7, wherein the blended image frame is derived from colorizing the visible-light image frame based on the ratio of the fluorescence image frame to one or more channels of the visible-light frame.

9. The method of claim 5, further comprising: displaying the blended image frame by adding the blended image frame to a video stream.

10. The method of claim 3, wherein the visible-light illumination source is pulsed.

11. The method of claim 10, wherein the pulsed visible-light illumination source is configured to include:
a plurality of primary visible-light illumination pulses for illuminating the tissue of the subject while the liquid crystal light shutter is in the open state; and
one or more compensating visible-light illumination pulses between each two neighboring primary visible-light illumination pulses.

12. The method of claim 11, wherein the liquid crystal light shutter is in the closed state during the one or more compensating visible-light illumination pulses.

13. The method of claim 10, further comprising:
while the fluorescence excitation illumination source is off and the liquid crystal light shutter is in the closed state, illuminating the tissue of the subject with ambient illumination to accumulate charge at the plurality of pixels of the global shutter imager;
reading a third set of accumulated charge at the plurality of pixels of the global shutter imager to produce a third set of imaging data; and
generating an ambient image frame based on the third set of imaging data.

14. The method of claim 13, wherein the ambient illumination comprises ambient illumination in the fluorescence emission band.

15. The method of claim 13, further comprising: subtracting the ambient image frame from the fluorescence image frame.

16. The method of claim 3, wherein the visible-light illumination source is continuous.

17. The method of claim 16, further comprising:
while the fluorescence excitation illumination source is off and the liquid crystal light shutter is in the closed state, illuminating the tissue of the subject with ambient illumination to accumulate charge at the plurality of pixels of the global shutter imager;
reading a third set of accumulated charge at the plurality of pixels of the global shutter imager to produce a third set of imaging data; and
generating an ambient image frame based on the third set of imaging data.

18. The method of claim 17, wherein the ambient illumination comprises ambient illumination in the fluorescence emission band.

19. The method of claim 17, further comprising: subtracting the ambient image frame from the fluorescence image frame.

20. A system for imaging tissue of a subject, the system comprising:
a fluorescence excitation illumination source,
a visible-light illumination source,
a liquid crystal light shutter configurable to be in an open state and a closed state, and an imager being configured for:
transitioning the liquid crystal light shutter to the closed state to prevent the global shutter imager from receiving visible light;
illuminating the tissue of the subject with the fluorescence excitation illumination source to accumulate charge at a plurality of pixels of the global shutter imager, while the liquid crystal light shutter is in the closed state;
reading a set of accumulated charge at the plurality of pixels of the global shutter imager to produce a set of imaging data; and
generating a fluorescence image frame based on the set of imaging data.
